(19) United States
(12) Patent Application Publication
(10) Pub. No.: US 2006/0247173 A1
(43) Pub. Date: Nov. 2, 2006

(54) Alpha-conotoxin peptides

(75) Inventors: Baldomero M. Olivera, Salt Lake City, UT (US); Richard T. Layer, Sandy, UT (US); Maren Watkins, Salt Lake City, UT (US); David R. Hillyard, Salt Lake City, UT (US); J. Michael McIntosh, Salt Lake City, UT (US); Robert Schoenfeld, Sacramento, CA (US); Jacob Scott Nielsen, Brigham City, UT (US); Robert M. Jones, Salt Lake City, UT (US)

Correspondence Address: ROTHWELL, FIGG, ERNST & MANBECK, P.C., 1425 K STREET, N.W., SUITE 800, WASHINGTON, DC, 20005, US

(73) Assignees: The University of Utah Research Foundation, Salt Lake City, UT (US); Cognetix, Inc., Salt Lake City, UT (US)

(21) Appl. No.: 11/482,148

(22) Filed: Jul. 7, 2006

Related U.S. Application Data
(63) Continuation of application No. 10/839,179, filed on May 6, 2004, now abandoned.
Application No. 10/839,179 is a division of application No. 09/908,741, filed on Jul. 20, 2001, now Pat. No. 6,855,805.
Application No. 09/908,741 is a continuation-in-part of application No. 09/488,799, filed on Jan. 21, 2000, now Pat. No. 6,268,473.
(60) Provisional application No. 60/116,881, filed on Jan. 22, 1999.
(60) Provisional application No. 60/116,882, filed on Jan. 22, 1999.

(51) Int. Cl. A61K 38/17; C07K 14/435

(52) U.S. Cl. 514/12; 530/324

(57) ABSTRACT

The invention relates to relatively short peptides (termed α-conotoxins herein), about 10-25 residues in length, which are naturally available in minute amounts in the venom of the cone snails or analogous to the naturally available peptides, and which preferably include two disulfide bonds. The α-conotoxins, as described herein, are useful for as neuromuscular blocking agents, such as muscle relaxants.

### CROSS-REFERENCE TO RELATED APPLICATIONS

The present application is a continuation of U.S. patent application Ser. No. 10/839,179 filed on 6 May 2004, which in turn is a division of U.S. patent application Ser. No. 09/908,741 filed on 20 Jul. 2001, which in turn is a continuation-in-part of U.S. patent application Ser. No. 09/488,799 filed on 21 Jan. 2000, now U.S. Pat. No. 6,268,473. Ser. No. 09/488,799 is related to and claims benefit under 35 USC § 119(e) to U.S. provisional patent application Ser. No. 60/116,881 filed on 22 Jan. 1999 and Ser. No. 60/116,882 filed on 22 Jan. 1999. Ser. No. 09/908,741 is related to and claims benefit under 35 USC § 119(e) to U.S. provisional patent application Ser. No. 60/219,407 filed on 20 Jul. 2000 and Ser. No. 60/221,557 filed on 28 Jul. 2000. Each of these applications is incorporated herein by reference.

This invention was made with Government support under Grant No. PO1 GM48677 awarded by the National Institute of General Medical Sciences, National Institutes of Health, Bethesda, Md. and under SBIR grant No. 1 R43 GM62064-01. The United States Government has certain rights in the invention.

### BACKGROUND OF THE INVENTION

The invention relates to relatively short peptides (termed α-conotoxins herein), about 10-25 residues in length, which are naturally available in minute amounts in the venom of the cone snails or analogous to the naturally available peptides, and which preferably include two disulfide bonds. The α-conotoxins, as described herein, are useful for as neuromuscular blocking agents, such as muscle relaxants.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

The predatory cone snails (Conus) have developed a unique biological strategy. Their venom contains relatively small peptides that are targeted to various neuromuscular receptors and may be equivalent in their pharmacological diversity to the alkaloids of plants or secondary metabolites of microorganisms. Many of these peptides are among the smallest nucleic acid-encoded translation products having defined conformations, and as such, they are somewhat unusual. Peptides in this size range normally equilibrate among many conformations. Proteins having a fixed conformation are generally much larger.

The cone snails that produce these peptides are a large genus of venomous gastropods comprising approximately 500 species. All cone snail species are predators that inject venom to capture prey, and the spectrum of animals that the genus as a whole can envenomate is broad. A wide variety of hunting strategies are used, however, every Conus species uses fundamentally the same basic pattern of envenomation.

Several peptides isolated from Conus venoms have been characterized. These include the α-, μ- and ω-conotoxins which target nicotinic acetylcholine receptors, muscle sodium channels, and neuronal calcium channels, respectively (Olivera et al., 1985). Conopressins, which are vasopressin analogs, have also been identified (Cruz et al. 1987). In addition, peptides named conantokins have been isolated from Conus geographus and Conus tulipa (Mena et al., 1990; Haack et al., 1990).

The α-conotoxins are small peptides highly specific for neuromuscular junction nicotinic acetylcholine receptors (Gray et al., 1981; Marshall and Harvey, 1990; Blount et al., 1992). The α-conotoxin peptides MI and GI are selective for the α/δ subunit interface of the neuromuscular junction nicotinic receptor over the α/γ subunit interface by >10,000 fold, while the α-conotoxin peptides EI and EIA bind both sites with equal affinity. However, none of these peptides show siginificant affinity for neuronal nicotinic receptors.

Various compounds having muscle relaxant properties are set forth in U.S. Pat. Nos. 4,190,674; 4,508,715; 4,761,418; 4,701,460; 4,179,507; 4,923,898; 5,015,741; and 5,260,337; as well as in Goodman and Gilman's The Pharmacological Basis of Therapeutics, Section II, especially Chapter 11, 7th Ed. (1985) and Physicians Desk Reference, 48 Ed., pp. 689, 758, 1362 and 1648 (1994).

Compounds having musculoskeletal relaxing properties include (1) agents acting in the central nervous system which are used to relieve pain associated with muscle contraction (e.g., 5-chlorobenzoxazolinone available as Parafon Forte DSC from McNeil Pharmaceutical), and (2) agents acting in the peripheral nervous system used primarily to induce muscle relaxation and hence reduce muscle contraction during anesthesia. The second group of muscle relaxants is subdivided into two groups: (i) non-depolarizing agents which inhibit the activation of muscle receptors (e.g., metocurarine iodide, d-tubocurarine, tubocurarine chloride, pancuronium, gallamine, diallytoiferine, toxiferine, atracurium besylate which is available as Tracrium from Burroughs-Wellcome Co., and vecuronium bromide which is available as Norcuron from Organon Inc.) and (ii) depolarizing agents which transiently activate muscle receptors and result in their blockade (e.g., decamethonium iodide, and succinylcholine chloride which is available as Anectine from Burroughs-Wellcome Co.). The effects of the depolarizing agents are manifested as fasciculations and flaccid paralysis which are observed to occur rapidly after their injection.

The effects of depolarizing agents (DA) and non-depolarizing agents (NDA) are separated based on their duration of action from ultrashort acting (e.g. for a depolarizing agent such as succinylcholine chloride) to intermediate (e.g. for a non-depolarizing agent such as atracurium besylate). Certain types of muscle relaxants are useful as neuromuscular blocking agents in clinical applications, and have found use as adjuvants to surgical anesthesia, in orthopedic surgical procedures and in facilitating endotracheal intubation procedures. Some of these compounds (e.g., succinylcholine chloride) are routinely used to provide muscle relaxation during Cesarean section procedures.

It is desirable for neuromuscular blocking agents to be locally acting and highly selective for binding to muscle nicotinic acetylcholine receptor sites. As such, when a patient is treated with anesthesia, the muscle relaxant is applied (e.g., intravenously or by injection), in order to cause the muscle to relax and hence minimize muscle contraction.

In anesthesia, neuromuscular blocking agents are used to provide skeletal muscular relaxation during surgery and during intubation of the trachea. All of the conventional nondepolarizing agents when used for producing skeletal muscle relaxation in surgery have a long duration of action e.g., 60 to 180 minutes in man. The depolarizing agents on the other hand provide muscle relaxation at dosages normally used for surgery which is less than the duration of action of nondepolarizing agents. For example, succinylcholine provides a short duration of action of about 5 to 15 minutes whereas decamethonium provides about 20 to 40 minutes duration of muscle relaxation. The long duration of action of nondepolarizing agents is unacceptable in many surgical procedures which take less than one hour because the patient is not generally fully recovered from their effects e.g., the patient may be unable to breathe adequately on his or her own.

Each nondepolarizing agent has inherent side-effects. For example, gallamine and pancuronium may cause tachycardia, d-tubocurarine and diallyltoxiferine may cause hypotension, and succinylcholine may cause fasciculations, myalgia, potassium release, cardiovascular effects, immunological reactions and malignant hyperthermia. While such drugs can be pharmacologically antagonized with anticholinesterase agents, this obviously necessitates the administration of a second drug which itself may have its own side effects e.g., bradycardia, gut spasm and bronchorrhea. Thus to overcome the aforementioned side-effects of the anticholinesterase agents, a third drug, an anticholinergic drug e.g., atropine must also be given.

With the use of depolarizing agents, there is no need to reverse the effects of the depolarizing agents, in certain patients the effects are much prolonged because of abnormal metabolism of the agent by the patient. The polarizing agents due to the mode of action which initially causes skeletal muscle contraction and stimulation of smooth muscles are also known to cause the following side-effects in certain instances; increased intraocular, and intragastric tension, cardiac arrhythmias, potassium release, and muscle pain. These side-effects caused by the depolarizing agents are not caused by the nondepolarizing agents. It is therefore clearly evident that a new neuromuscular blocking agent having the relatively few side-effects and the reversibility of the nondepolarizing agents yet being of considerably shorter i.e., intermediate, duration of action is needed.

It is desired to provide a compound useful as a muscle relaxant. In particular, it is desired to provide an antagonist which has activity at relatively low concentrations as a neuromuscular blocking agent. It is also desired to achieve muscle relaxation at concentrations of agonist that are devoid of any ganglionic effects (e.g., so as to not exhibit side effects such as those associated with interaction with cardiovascular sites). As such, it is desired to provide muscle relaxant compositions and methods for providing muscle relaxation. Finally, it is desired to identify additional α-conotoxin peptides for use as neuromuscular blocking agents.

### SUMMARY OF THE INVENTION

The invention relates to relatively short peptides (termed α-conotoxins herein), about 10-25 residues in length, which are naturally available in minute amounts in the venom of the cone snails or analogous to the naturally available peptides, and which preferably include two disulfide bonds. The α-conotoxins, as described herein, are useful for as neuromuscular blocking agents, such as muscle relaxants, for treating benign essential blepharospasm and other forms of focal dystonia and for anti-wrinkle use.

More specifically, the present invention is directed to the neuromuscular blocking use of α-conotoxin peptides of two classes, namely, (a) α3/5 or α3/6 and (b) α4/7, as described herein. The first class of α-conotoxin peptides has the general formula I:

Xaa₁-Xaa₂-Xaa₃-Xaa₄-Cys-Cys-Xaa₅-Xaa₆-Xaa₇-Cys-Xaa₈-Xaa₉-Xaa₁₀-Xaa₁₁-Xaa₁₂-Xaa₁₃-Cys-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅ (SEQ ID NO:1), wherein Xaa₁ is des-Xaa₁ or Gly; Xaa₂ is des-Xaa₂, Asn, Arg, Asp, Ser, Thr, Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₃ is des-Xaa₃, Gly, Glu or γ-carboxy-Glu (Gla); Xaa₄, is des-Xaa₄, Glu, Gla, Gln, pyro-Glu, Arg, Ile Tyr, mono-halo-Tyr, di-halo-Tyr, O-sulpho-Tyr, O-phospho-Tyr, nitro-Tyr, Cys, His, halo-His, any unnatural hydroxy containing amino acid (such as 4-hydroxymethyl-Phe, 4-hydroxyphenyl-Gly, 2,6-dimethyl-Tyr and 5-amino-Tyr), Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₅ is His, Asn or halo-His; Xaa₆ is Pro or hyroxy-Pro; Xaa₇ is Ala, Gly, Ser or Thr; Xaa₈ is Gly or Ala; Xaa₉ is Arg, Lys, Pro, hydroxy-Pro, Gly, Gln, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₁₀ is His, halo-His, Asn, Lys, Tyr, mono-halo-Tyr, di-halo-Tyr, O-sulpho-Tyr, O-phospho-Tyr, nitro-Tyr, N-methy-Lys, N,N-dimethyl-Lys, N,N,N-trimethyl-Lys, Arg, homoarginine, ornithine or any unnatural basic amino acid (such as N-1-(2-pyrazolinyl)-Arg); Xaa₁₁ is Tyr, Phe, mono-halo-Tyr, di-halo-Tyr, O-sulpho-Tyr, O-phospho-Tyr, nitro-Tyr, any unnatural hydroxy containing amino acid (such as 4-hydroxymethyl-Phe, 4-hydroxyphenyl-Gly, 2,6-dimethyl-Tyr and 5-amino-Tyr), Trp (D or L), halo-Trp, neo-Trp, or any unnatural aromatic amino acid (such as nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc); Xaa₁₂ is Ile, Ser, Thr, Asp, Gly, Asn, Glu, Gla or Val; Xaa₁₃ is des-Xaa₁₃, Lys, Arg, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₁₄ is des-Xaa₁₄, Gly, Lys, Arg, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₁₅ is des-Xaa₁₅, Gly, Thr, Ser, His, halo-His, Lys, Arg, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₁₆ is des-Xaa₁₆, Ser or Thr; Xaa₁₇ is des-Xaa₁₇ or Cys; Xaa₁₈ is des-Xaa₁₈, Ser or Thr; Xaa₁₉ is des-Xaa₁₉, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₂₀ is des-Xaa₂₀, Thr, Ser, Pro or hydroxy-Pro; Xaa₂₁ is des-Xaa₂₁, Leu, Ser or Thr; Xaa₂₂ is des-Xaa₂₂, Glu or Gla; Xaa₂₃ is des-Xaa₂₃, Pro or hydroxy-Pro; Xaa₂₄ is des-Xaa₂₄, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg); and Xaa₂₅ is des-Xaa₂₅, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg). The C-terminus may contain a free carboxyl group or an amide group, preferably an amide group. The halo is chlorine, bromine or iodine, preferably iodine for Tyr and bromine for Trp. The Cys residues may be in D or L configuration and may optionally be substituted with homocysteine.

Useful peptides include GI (Gray et al., 1981), GIA (Gray et al., 1981), GII (Gray et al., 1981), MI (McIntosh et al., 1982), SI (Zafaralla et al., 1988), SIA (Myers et al., 1991), SIB (same as SI, except further contains Glu at N-terminus), SII (Olivera et al., 1996), SIIA (Olivera et al., 1996), R1 (same as G1, except Tyr for Lys), R1.3 (below), R1.4 (below), 5 m1.1 (below), S11 (below), S2 (below); GIB (same as R1); MnII (below); A1.2 (below); A1.3 (below); A1.7 (below); A1.8 (below); Ay1.1 (below); Ay1.1a (below); M1.1 (below); M1.3 (below); M1.4 (below); M1.5 (below); O1.3 (below); S1.3 (below); Sa (below). Additional useful peptides are analogs of MI and GI as described below.

The second class of α-conotoxin peptides has the general formula II:

Xaa₁-Xaa₂-Xaa₃-Cys-Cys-Xaa₄-Xaa₅-Xaa₆-Xaa₇-Cys-Xaa₈-Xaa₉-Xaa₁₀-Xaa₁₁-Xaa₆-Xaa₁₂-Ile-Cys-Xaa₁₃-Xaa₁₄-Xaa₁₅ (SEQ ID NO:2), wherein, Xaa₁ is des-Xaa₁, Arg, Ser, Thr, Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₂ is des-Xaa₂, Asp, Gly, Leu, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₃ is des-Xaa₃, Pro, hydroxy-Pro, Ala, Gly or Leu; Xaa₄ is Tyr, Ser, Thr, mono-halo-Tyr, di-halo-Tyr, O-sulpho-Tyr, O-phospho-Tyr, nitro-Tyr or any unnatural hydroxy containing amino acid (such as 4-hydroxymethyl-Phe, 4-hydroxyphenyl-Gly, 2,6-dimethyl-Tyr and 5-amino-Tyr); Xaa₅ is His, Asn, Ile, Tyr, halo-His, mono-halo-Tyr, di-halo-Tyr, O-sulpho-Tyr, O-phospho-Tyr or nitro-Tyr; Xaa₆ is Pro or hydroxy-Pro; Xaa₇ is Thr, Ala, Val, Ser, Pro or hydroxy-Pro; Xaa₈ is Asn, Thr, Ser, Lys, Arg, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₉ is Met, Val, Ala, Leu or Ile; Xaa₁₀ is Ser, Thr, Asn, His or halo-His; Xaa₁₁ is Asn, Tyr, mono-halo-Tyr, di-halo-Tyr, O-sulpho-Tyr, O-phospho-Tyr, nitro-Tyr, or any unnatural hydroxy containing amino acid (such as 4-hydroxymethyl-Phe, 4-hydroxyphenyl-Gly, 2,6-dimethyl-Tyr and 5-amino-Tyr); Xaa₁₂ is Glu, γ-carboxy-Glu (Gla), Gln or Asp; Xaa₁₃ is des-Xaa₁₃ or Gly; Xaa₁₄ is des-Xaa₁₄ or Gly; and Xaa₁₅ is des-Xaa₁₅, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg). The C-terminus may contain a free carboxyl group or an amide group, preferably an amide group. The halo is preferably chlorine or iodine, more preferably iodine. The Cys residues may be in D or L configuration and may optionally be substituted with homocysteine.

Useful peptides include E1 (U007; Olivera et al., 1996), EIA (U008; Olivera et al., 1996), P1.2 (below), P1.3 (below), S11.4 (below), S11.4A (below); S11.8 (below) and Ta (below).

The present invention is also directed to novel specific α-conotoxin peptides of class I having the formulas:

Xaa₁-Cys-Cys-Asn-Xaa₂-Ala-Cys-Gly-Arg-His-Xaa₃-Ser-Cys-Xaa₄-Gly (SEQ ID NO:3);

Asn-Gly-Arg-Cys-Cys-His-Xaa₂-Ala-Cys-Gly-Xaa₄-His-Phe-Ser-Cys (SEQ ID NO:4);

Gly-Arg-Gly-Arg-Cys-Cys-His-Xaa₂-Ala-Cys-Gly-Xaa₂-Asn-Xaa₃-Ser-Cys (SEQ ID NO:5);

Cys-Cys-His-Xaa₂-Ala-Cys-Gly-Arg-Xaa₄-Xaa₃-Asn-Cys (SEQ ID NO:6);

Cys-Cys-Cys-Asn-Xaa₂-Ala-Cys-Gly-Xaa₂-Asn-Xaa₃-Gly-Cys-Gly-Thr-Ser-Cys-Ser-Arg-Xaa₂-Ser-Xaa₁-Xaa₂-Arg-Arg (SEQ ID NO:7);

Asn-Gly-His-Cys-Cys-His-Xaa₂-Ala-Cys-Gly-Gly-Xaa₄-Xaa₃-Val-Xaa₄-Cys (SEQ ID NO:8);

Asn-Gly-Arg-Cys-Cys-His-Xaa₂-Ala-Cys-Gly-Gly-Xaa₄-Xaa₃-Val-Xaa₄-Cys (SEQ ID NO:9);

Asn-Gly-Arg-Cys-Cys-His-Xaa₂-Ala-Cys-Gly-Xaa₄-His-Phe-Ile-Cys (SEQ ID NO:10);

Asn-Gly-Arg-Cys-Cys-His-Xaa₂-Ala-Cys-Gly-Xaa₄-His-Phe-Ser-Cys (SEQ ID NO:11);

Asn-Gly-Arg-Cys-Cys-His-Xaa₂-Ser-Cys-Gly-Arg-Xaa₄-Xaa₃-Asn-Cys (SEQ ID NO:12);

Asn-Gly-Arg-Cys-Cys-His-Xaa₂-Ala-Cys-Ala-Arg-Xaa₄-Xaa₃-Asn-Cys (SEQ ID NO:13);

Asn-Xaa₁-Arg-Cys-Cys-His-Xaa₂-Ala-Cys-Ala-Arg-Xaa₄-Xaa₃-Asn-Cys (SEQ ID NO:14);

Asp-Gly-Arg-Cys-Cys-His-Xaa₂-Ala-Cys-Gly-Gln-Asn-Xaa₃-Ser-Cys (SEQ ID NO:15);

Asp-Gly-Arg-Cys-Cys-His-Xaa₂-Ala-Cys-Ala-Xaa₄-His-Phe-Asn-Cys (SEQ ID NO:16);

Asn-Gly-Arg-Cys-Cys-His-Xaa₂-Ala-Cys-Ala-Xaa₄-Asn-Xaa₃-Ser-Cys (SEQ ID NO:17);

Asn-Gly-Arg-Cys-Cys-His-Xaa₂-Ala-Cys-Ala-Arg-Xaa₄-Xaa₃-Ser-Cys (SEQ ID NO:18);

Xaa₅-Cys-Cys-Asn-Xaa₂-Ala-Cys-Gly-Xaa₂-Xaa₄-Xaa₃-Ser-Cys (SEQ ID NO:19);

Xaa₅-Cys-Cys-His-Xaa₂-Ala-Cys-Gly-Xaa₄-Xaa₄-Xaa₃-Asn-Cys (SEQ ID NO:20); and

Ser-Gly-Arg-Cys-Cys-His-Xaa₂-Ala-Cys-Gly-Arg-Xaa₄-Xaa₃-Asn-Cys (SEQ ID NO:21),

wherein Xaa₁ is Glu or γ-carboxy-glutamate (Gla); Xaa₂ is Pro or hydroxy-Pro; Xaa₃ is Tyr, mono-halo-Tyr, di-halo-Tyr, O-sulpho-Tyr, O-phospho-Tyr or nitro-Tyr; Xaa₄ is Lys, N-methyl-Lys, N,N-dimethyl-Lys or N,N,N-trimethyl-Lys; Xaa₅ is Gln or pyro-Glu; and the C-terminus contains a carboxyl or amide group, preferably an amide group. The halo is preferably chlorine or iodine, more preferably iodine. In addition, the His residues may be substituted with halo-His; the Arg residues may be substituted by Lys, ornithine, homoargine, N-methy-Lys, N,N-dimethyl-Lys, N,N,N-trimethyl-Lys or any unnatural basic amino acid (such as N-1-(2-pyrazolinyl)-Arg); the Lys residues may be substituted by Arg, ornithine, homoargine, N-methy-Lys, N,N-dimethyl-Lys, N,N,N-trimethyl-Lys or any unnatural basic amino acid (such as N-1-(2-pyrazolinyl)-Arg); the Tyr residues may be substituted with ¹²⁵I-Tyr or any unnatural hydroxy containing amino acid (such as 4-hydroxymethyl-Phe, 4-hydroxyphenyl-Gly, 2,6-dimethyl-Tyr and 5-amino-Tyr); the Ser residues may be substituted with Thr; the Thr residues may be substituted with Ser; and the Phe residues may be substituted with any unnatural aromatic amino acid (such as nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc).

More specifically, the present invention is directed to the following α-conotoxin peptides of class I:

R1.3: SEQ ID NO:3, wherein Xaa₁ is Glu, Xaa₂ is Pro, Xaa₃ is Tyr and Xaa₄ is Lys;

R1.4: SEQ ID NO:4, wherein Xaa₂ is Pro and Xaa₄ is Lys;

Sm1.1: SEQ ID NO:5, wherein Xaa₂ is Pro and Xaa₃ is Tyr;

S11: SEQ ID NO:6, wherein Xaa₂ is Pro, Xaa₃ is Tyr and Xaa₄ is Lys;

S2: SEQ ID NO:7, wherein Xaa₁ is Glu, Xaa₂ is Pro and Xaa₃ is Tyr;

MnII: SEQ ID NO:8, wherein Xaa₂ is Pro, Xaa₃ is Tyr and Xaa₄ is Lys;

A1.2: SEQ ID NO:9, wherein Xaa₂ is Pro, Xaa₃ is Tyr and Xaa₄ is Lys;

A1.3: SEQ ID NO:10, wherein Xaa₂ is Pro and Xaa₄ is Lys;

A1.7: SEQ ID NO:11, wherein Xaa₂ is Pro and Xaa₄ is Lys;

A1.8: SEQ ID NO:12, wherein Xaa₂ is Pro and Xaa₄ is Lys;

Ay1.1: SEQ ID NO:13, wherein Xaa₂ is Pro, Xaa₃ is Tyr and Xaa₄ is Lys;

Ay1.1a: SEQ ID NO:14, wherein Xaa₁ is Glu, Xaa₂ is Pro, Xaa₃ is Tyr and Xaa₄ is Lys;

M1.1: SEQ ID NO:15, wherein Xaa₂ is Pro and Xaa₃ is Tyr;

M1.3: SEQ ID NO:16, wherein Xaa₂ is Pro and Xaa₄ is Lys;

M1.4: SEQ ID NO:17, wherein Xaa₂ is Pro, Xaa₃ is Tyr and Xaa₄ is Lys;

M1.5: SEQ ID NO:18, wherein Xaa₂ is Pro, Xaa₃ is Tyr and Xaa₄ is Lys;

O1.3: SEQ ID NO:19, wherein Xaa₂ is Pro, Xaa₃ is Tyr, Xaa₄ is Lys and Xaa₅ is Gln;

S1.3: SEQ ID NO:20, wherein Xaa₂ is Pro, Xaa₃ is Tyr, Xaa₄ is Lys and Xaa₅ is Gln; and

Sa: SEQ ID NO:21, wherein Xaa₂ is Pro, Xaa₃ is Tyr and Xaa₄ is Lys.

The C-terminus is preferably amidated in each of these specific peptides.

The present invention is further directed to MI and GI analogs having the formulas:

MI[K10Q]: Gly-Arg-Cys-Cys-His-Pro-Ala-Cys-Gly-Gln-Asn-Tyr-Ser-Cys (SEQ ID NO:102);

MI[K10E]: Gly-Arg-Cys-Cys-His-Pro-Ala-Cys-Gly-Glu-Asn-Tyr-Ser-Cys (SEQ ID NO:103);

MI[K10Q, N11Q]: Gly-Arg-Cys-Cys-His-Pro-Ala-Cys-Gly-Gln-Gln-Tyr-Ser-Cys (SEQ ID NO:104);

MI[H5N, K10Q]: Gly-Arg-Cys-Cys-Asn-Pro-Ala-Cys-Gly-Gln-Asn-Tyr-Ser-Cys (SEQ ID NO:105);

MI[K10N]: Gly-Arg-Cys-Cys-His-Pro-Ala-Cys-Gly-Asn-Asn-Tyr-Ser-Cys (SEQ ID NO:106);

desG1-MI[K10Q, N11Q]: Arg-Cys-Cys-His-Pro-Ala-Cys-Gly-Gln-Gln-Tyr-Ser-Cys (SEQ ID NO:107);

MI[K10Q, S13D]: Gly-Arg-Cys-Cys-His-Pro-Ala-Cys-Gly-Gln-Asn-Tyr-Asp-Cys (SEQ ID NO:108);

MI[K10homoSer]: Gly-Arg-Cys-Cys-His-Pro-Ala-Cys-Gly-Xaa-Asn-Tyr-Ser-Cys (SEQ ID NO:109), where Xaa is homoserine;

desG1-MI[R2E, K10Q]: Glu-Cys-Cys-His-Pro-Ala-Cys-Gly-Gln-Asn-Tyr-Ser-Cys (SEQ ID NO:110);

desG1/R2-MI[K10Q]:Cys-Cys-His-Pro-Ala-Cys-Gly-Gln-Asn-Tyr-Ser-Cys (SEQ ID NO:111);

MI[K10Q, Y12F]: Gly-Arg-Cys-Cys-His-Pro-Ala-Cys-Gly-Gln-Asn-Phe-Ser-Cys (SEQ ID NO:112);

MI[K10Q, S13K]: Gly-Arg-Cys-Cys-His-Pro-Ala-Cys-Gly-Gln-Asn-Tyr-Lys-Cys (SEQ ID NO:113);

MI[R2E, K10Q]: Gly-Glu-Cys-Cys-His-Pro-Ala-Cys-Gly-Gln-Asn-Tyr-Ser-Cys (SEQ ID NO:114);

MI[C4E, K10Q, C14K]: Gly-Arg-Cys-Glu-His-Pro-Ala-Cys-Gly-Gln-Asn-Tyr-Ser-Lys (SEQ ID NO:115), wherein Glu4 and Lys14 form a lactam bridge in place of the disulfide bridge in the native MI;

MI[C4E, K10N, C14K]: Gly-Arg-Cys-Glu-His-Pro-Ala-Cys-Gly-Asn-Asn-Tyr-Ser-Lys (SEQ ID NO:116), wherein Glu4 and Lys 14 form a lactam bridge in place of the disulfide bridge in the native MI;

MI[C4D, K10Q, C14K]: Gly-Arg-Cys-Asp-His-Pro-Ala-Cys-Gly-Gln-Asn-Tyr-Ser-Lys (SEQ ID NO:117), wherein Asp4 and Lys 14 form a lactam bridge in place of the disulfide bridge in the native MI;

MI[C4D, K10N, C14K]: Gly-Arg-Cys-Asp-His-Pro-Ala-Cys-Gly-Asn-Asn-Tyr-Ser-Lys (SEQ ID NO:118), wherein Asp4 and Lys 14 form a lactam bridge in place of the disulfide bridge in the native MI;

GI[R9Q]: Glu-Cys-Cys-Asn-Pro-Ala-Cys-Gly-Gln-His-Tyr-Ser-Cys (SEQ ID NO:119);

GI[R9N]: Glu-Cys-Cys-Asn-Pro-Ala-Cys-Gly-Asn-His-Tyr-Ser-Cys (SEQ ID NO:120);

GI[C3E, C13K]: Glu-Cys-Glu-Asn-Pro-Ala-Cys-Gly-Arg-His-Tyr-Ser-Lys (SEQ ID NO:121), wherein Glu3 and Lys 13 form a lactam bridge in place of the disulfide bridge in the native GI;

GI[C3E, R9Q, C13K]: Glu-Cys-Glu-Asn-Pro-Ala-Cys-Gly-Gln-His-Tyr-Ser-Lys (SEQ ID NO:122), wherein Glu3 and Lys 13 form a lactam bridge in place of the disulfide bridge in the native GI;

GI[C3E, R9N, C13K]: Glu-Cys-Glu-Asn-Pro-Ala-Cys-Gly-Asn-His-Tyr-Ser-Lys (SEQ ID NO:123), wherein Glu3 and Lys 13 form a lactam bridge in place of the disulfide bridge in the native GI;

GI[C3D, R9Q, C13K]: Glu-Cys-Asp-Asn-Pro-Ala-Cys-Gly-Gln-His-Tyr-Ser-Lys (SEQ ID NO:124), wherein Asp3 and Lys 13 form a lactam bridge in place of the disulfide bridge in the native GI; and

GI[C3D, R9N, C13K]: Glu-Cys-Asp-Asn-Pro-Ala-Cys-Gly-Asn-His-Tyr-Ser-Lys (SEQ ID NO:125), wherein Asp3 and Lys13 form a lactam bridge in place of the disulfide bridge in the native GI.

The C-terminus is preferably amidated in each of these specific peptides.

The present invention is also directed to novel specific α-conotoxin peptides of class II having the formulas:

Arg-Asp-Xaa₂-Cys-Cys-Ser-Asn-Xaa₂-Val-Cys-Thr-Val-His-Asn-Xaa₂-Gln-Ile-Cys (SEQ ID NO:22);

Arg-Ala-Cys-Cys-Ser-Xaa₃-Xaa₂-Xaa₂-Cys-Asn-Val-Asn-Xaa₃-Xaa₂-Xaa₁-Ile-Cys (SEQ ID NO:23);

Gly-Gly-Cys-Cys-Ser-Xaa₃-Xaa₂-Xaa₂-Cys-Asn-Val-Ser-Xaa₃-Xaa₂-Xaa₁-Ile-Cys (SEQ ID NO:24);

Cys-Cys-Ser-Xaa₃-Xaa₂-Xaa₂-Cys-Asn-Val-Ser-Xaa₃-Xaa₂-Xaa₁-Ile-Cys (SEQ ID NO:25);

Ala-Cys-Cys-Ser-Xaa₃-Xaa₂-Xaa₂-Cys-Asn-Val-Asn-Xaa₃-Xaa₂-Xaa₁-Ile-Cys-Gly-Gly-Arg (SEQ ID NO:26); and

Ser-Leu-Leu-Cys-Cys-Thr-Ile-Xaa₂-Ser-Cys-Xaa₄-Ala-Ser-Xaa₃-Xaa₂-Asp-Ile-Cys (SEQ ID NO:27),

wherein Xaa₁ is Glu or γ-carboxy-Glu (Gla); Xaa₂ is Pro or hydroxy-Pro; Xaa₃ is Tyr, mono-halo-Tyr, di-halo-Tyr, O-sulpho-Tyr, O-phospho-Tyr or nitro-Tyr; Xaa₄ is Lys, N-methyl-Lys, N,N-dimethyl-Lys or N,N,N-trimethyl-Lys; and the C-terminus contains a carboxyl or amide group, preferably an amide group. The halo is preferably chlorine or iodine, more preferably iodine. In addition, the His residues may be substituted with halo-His; the Arg residues may be substituted by Lys, ornithine, homoargine, N-methy-Lys, N,N-dimethyl-Lys, N,N,N-trimethyl-Lys or any unnatural basic amino acid (such as N-1-(2-pyrazolinyl)-Arg); the Lys residues may be substituted by Arg, ornithine, homoargine, N-methy-Lys, N,N-dimethyl-Lys, N,N,N-trimethyl-Lys or any unnatural basic amino acid (such as N-1-(2-pyrazolinyl)-Arg); and the Tyr residues may be substituted with ¹²⁵I-Tyr or any unnatural hydroxy containing amino acid (such as 4-hydroxymethyl-Phe, 4-hydroxyphenyl-Gly, 2,6-dimethyl-Tyr and 5-amino-Tyr).

More specifically, the present invention is directed to the following α-conotoxin peptides of class II:

P1.2: SEQ ID NO:22, wherein Xaa₂ is Pro;

P1.3: SEQ ID NO:23, wherein Xaa₁ is Glu, Xaa₂ is Pro and Xaa₃ is Tyr;

S11.4: SEQ ID NO:24, wherein Xaa₁ is Glu, Xaa₂ is Pro and Xaa₃ is Tyr;

S11.4A: SEQ ID NO:25, wherein Xaa₁ is Glu, Xaa₂ is Pro and Xaa₃ is Tyr;

S11.8: SEQ ID NO:26, wherein Xaa₁ is Glu, Xaa₂ is Pro and Xaa₃ is Tyr; and

Ta: SEQ ID NO:27, wherein Xaa₂ is Pro, Xaa₃ is Tyr and Xaa₄ is Lys.

The C-terminus is preferably amidated in each of these specific peptides.

Examples of synthetic aromatic amino acid include, but are not limited to, such as nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also web address amino-acids. com), incorporated herein by reference, by and available from RSP Amino Acid Analogues, Inc., Worcester, Mass. Examples of synthetic acid amino acids include those derivatives bearing acidic functionality, including carboxyl, phosphate, sulfonate and synthetic tetrazolyl derivatives such as described by Omstein et al. (1993) and in U.S. Pat. No. 5,331,001, each incorporated herein by reference.

Optionally, in the peptides of general formulas I and II and the specific peptides and analogs described above, the Asn residues may be modified to contain an N-glycan and the Ser and Thr residues may be modified to contain an O-glycan. In accordance with the present invention, a glycan shall mean any N-, S- or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is beta and 1-4 or 1-3, preferably 1-3. The linkage between the glycan and the amino acid may be alpha or beta, preferably alpha and is 1-.

Core O-glycans have been described by Van de Steen et al. (1998), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. Ser. No. 09/420,797, filed 19 Oct. 1999 (now U.S. Pat. No. 6,369,193) and in PCT Application No. PCT/US99/24380, filed 19 Oct. 1999 (PCT Published Application No. WO 00/23092), each incorporated herein by reference. A preferred glycan is Gal(β1→3)GalNAc(α1→).

Optionally, in the above peptides, pairs of Cys residues may be replaced pairwise with isoteric lactam or ester-thioether replacements, such as Ser/(Glu or Asp), Lys/(Glu or Asp) or Cys/Ala combinations. Sequential coupling by known methods (Barnay et al., 2000; Hruby et al., 1994; Bitan et al., 1997) allows replacement of native Cys bridges with lactam bridges. Thioether analogs may be readily synthesized using halo-Ala residues commercially available from RSP Amino Acid Analogues.

The present invention is further directed to derivatives of the above peptides and peptide derivatives which are acylic permutations in which the cyclic permutants retain the native bridging pattern of native toxin. See Craik et al. (2001).

The present invention is further directed to propeptides and nucleic acid sequences encoding the propeptides or peptides as described in further detail herein.

### BRIEF DESCRIPTION OF THE FIGURES

FIG. 1 shows onset and recovery time of neuromuscular block for different doses (87, 100 or 150 μg/kg) of the α-conotoxin peptide MI.

FIG. 2 shows onset and recovery time of neuromuscular block for different doses (180, 217 or 250 μg/kg) of α-conotoxin peptide GI

FIG. 3 shows dose response curves for the α-conotoxin peptides MI ( [symbol] ) and GI ( [symbol] ).

FIG. 4 shows onset and recovery time of neuromuscular block for different doses (18.76, 28.125, 37.5, 75 or 150 μg/kg) of the α-conotoxin peptide mono-iodo-Tyr₁₂-MI.

FIG. 5 shows onset and recovery time of neuromuscular block for different doses (125, 137.5 or 150 μg/kg) of the α-conotoxin peptide di-iodo-Tyr₁₂-MI.

FIG. 6 shows dose response curve for the α-conotoxin peptide mono-iodo-Tyr₁₂-MI.

FIG. 7 shows dose response curve for the α-conotoxin peptide di-iodo-Tyr₁₂-MI.

### DETAILED DESCRIPTION OF THE PREFERRED EMBODIMENT

The invention relates to relatively short peptides (termed α-conotoxins herein), about 10-25 residues in length, which are naturally available in minute amounts in the venom of the cone snails or analogous to the naturally available peptides, and which preferably include two disulfide bonds. The α-conotoxins, as described herein, are useful for as neuromuscular blocking agents, such as muscle relaxants, for treating benign essential blepharospasm and other forms of focal dystonia and for anti-wrinkle use.

In one aspect, the present invention relates to a method for providing relaxation of muscle. The method involves administering to a patient an effective amount of an α-conotoxin peptide having the general formula set forth above. Exemplary methods involve administering to a patient an effective amount of MI, GI, EI, mono-iodo-MI (Tyr₁₂ of MI having an iodine) or di-iodo-MI (Tyr₁₂ of MI having two iodines).

The present invention, in another aspect, relates to a pharmaceutical composition comprising an effective amount of an α-conotoxin peptide having the general formula set forth above. Such a pharmaceutical composition has the capability of acting as a neuromuscular non-depolarizing agent, and hence has the capability of acting as a muscle relaxant. Exemplary pharmaceutical compositions acting as neuromuscular non-depolarizing muscle relaxants include as an active ingredient MI, GI, EI, mono-iodo-MI or di-iodo-MI.

The α-conotoxin peptides described herein are sufficiently small to be chemically synthesized. General chemical syntheses for preparing the foregoing α-conotoxin peptides are described hereinafter. Various ones of the α-conotoxin peptides can also be obtained by isolation and purification from specific Conus species using the technique described in U.S. Pat. No. 4,447,356 (Olivera et al., 1984), the disclosure of which is incorporated herein by reference.

Although the α-conotoxin peptides of the present invention can be obtained by purification from cone snails, because the amounts of α-conotoxin peptides obtainable from individual snails are very small, the desired substantially pure α-conotoxin peptides are best practically obtained in commercially valuable amounts by chemical synthesis using solid-phase strategy. For example, the yield from a single cone snail may be about 10 micrograms or less of α-conotoxin peptide. By “substantially pure” is meant that the peptide is present in the substantial absence of other biological molecules of the same type; it is preferably present in an amount of at least about 85% purity and preferably at least about 95% purity. Chemical synthesis of biologically active α-conotoxin peptides depends of course upon correct determination of the amino acid sequence.

The α-conotoxin peptides can also be produced by recombinant DNA techniques well known in the art. Such techniques are described by Sambrook et al. (1989). The peptides produced in this manner are isolated, reduced if necessary, and oxidized to form the correct disulfide bonds.

One method of forming disulfide bonds in the conantokin peptides of the present invention is the air oxidation of the linear peptides for prolonged periods under cold room temperatures or at room temperature. This procedure results in the creation of a substantial amount of the bioactive, disulfide-linked peptides. The oxidized peptides are fractionated using reverse-phase high performance liquid chromatography (HPLC) or the like, to separate peptides having different linked configurations. Thereafter, either by comparing these fractions with the elution of the native material or by using a simple assay, the particular fraction having the correct linkage for maximum biological potency is easily determined. However, because of the dilution resulting from the presence of other fractions of less biopotency, a somewhat higher dosage may be required.

The peptides are synthesized by a suitable method, such as by exclusively solid-phase techniques, by partial solid-phase techniques, by fragment condensation or by classical solution couplings.

In conventional solution phase peptide synthesis, the peptide chain can be prepared by a series of coupling reactions in which constituent amino acids are added to the growing peptide chain in the desired sequence. Use of various coupling reagents, e.g., dicyclohexylcarbodiimide or diisopropylcarbonyldimidazole, various active esters, e.g., esters of N-hydroxyphthalimide or N-hydroxy-succinimide, and the various cleavage reagents, to carry out reaction in solution, with subsequent isolation and purification of intermediates, is well known classical peptide methodology. Classical solution synthesis is described in detail in the treatise, “Methoden der Organischen Chemie (Houben-Weyl): Synthese von Peptiden,” (1974). Techniques of exclusively solid-phase synthesis are set forth in the textbook, “Solid-Phase Peptide Synthesis,” (Stewart and Young, 1969), and are exemplified by the disclosure of U.S. Pat. No. 4,105,603 (Vale et al., 1978). The fragment condensation method of synthesis is exemplified in U.S. Pat. No. 3,972,859 (1976). Other available syntheses are exemplified by U.S. Pat. Nos. 3,842,067 (1974) and 3,862,925 (1975). The synthesis of peptides containing γ-carboxyglutamic acid residues is exemplified by Rivier et al. (1987), Nishiuchi et al. (1993) and Zhou et al. (1996).

Common to such chemical syntheses is the protection of the labile side chain groups of the various amino acid moieties with suitable protecting groups which will prevent a chemical reaction from occurring at that site until the group is ultimately removed. Usually also common is the protection of an α-amino group on an amino acid or a fragment while that entity reacts at the carboxyl group, followed by the selective removal of the α-amino protecting group to allow subsequent reaction to take place at that location. Accordingly, it is common that, as a step in such a synthesis, an intermediate compound is produced which includes each of the amino acid residues located in its desired sequence in the peptide chain with appropriate side-chain protecting groups linked to various ones of the residues having labile side chains.

As far as the selection of a side chain amino protecting group is concerned, generally one is chosen which is not removed during deprotection of the α-amino groups during the synthesis. However, for some amino acids, e.g., His, protection is not generally necessary. In selecting a particular side chain protecting group to be used in the synthesis of the peptides, the following general rules are followed: (a) the protecting group preferably retains its protecting properties and is not split off under coupling conditions, (b) the protecting group should be stable under the reaction conditions selected for removing the α-amino protecting group at each step of the synthesis, and (c) the side chain protecting group must be removable, upon the completion of the synthesis containing the desired amino acid sequence, under reaction conditions that will not undesirably alter the peptide chain.

It should be possible to prepare many, or even all, of these peptides using recombinant DNA technology. However, when peptides are not so prepared, they are preferably prepared using the Merrifield solid-phase synthesis, although other equivalent chemical syntheses known in the art can also be used as previously mentioned. Solid-phase synthesis is commenced from the C-terminus of the peptide by coupling a protected α-amino acid to a suitable resin. Such a starting material can be prepared by attaching an α-amino-protected amino acid by an ester linkage to a chloromethylated resin or a hydroxymethyl resin, or by an amide bond to a benzhydrylamine (BHA) resin or para-methylbenzhydrylamine (MBHA) resin. Preparation of the hydroxymethyl resin is described by Bodansky et al. (1966). Chloromethylated resins are commercially available from Bio Rad Laboratories (Richmond, Calif.) and from Lab. Systems, Inc. The preparation of such a resin is described by Stewart and Young (1969). BHA and MBHA resin supports are commercially available, and are generally used when the desired polypeptide being synthesized has an unsubstituted amide at the C-terminus. Thus, solid resin supports may be any of those known in the art, such as one having the formulae —O—CH₂-resin support, —NH BHA resin support, or —NH-MBHA resin support. When the unsubstituted amide is desired, use of a BHA or MBHA resin is preferred, because cleavage directly gives the amide. In case the N-methyl amide is desired, it can be generated from an N-methyl BHA resin. Should other substituted amides be desired, the teaching of U.S. Pat. No. 4,569,967 (Kornreich et al., 1986) can be used, or should still other groups than the free acid be desired at the C-terminus, it may be preferable to synthesize the peptide using classical methods as set forth in the Houben-Weyl text (1974).

The C-terminal amino acid, protected by Boc or Fmoc and by a side-chain protecting group, if appropriate, can be first coupled to a chloromethylated resin according to the procedure set forth in K. Horiki et al. (1978), using KF in DMF at about 60° C. for 24 hours with stirring, when a peptide having free acid at the C-terminus is to be synthesized. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups may be used as described in Schroder & Lubke (1965).

After removal of the α-amino-protecting group, the remaining α-amino- and side chain-protected amino acids are coupled step-wise in the desired order to obtain the intermediate compound defined hereinbefore, or as an alternative to adding each amino acid separately in the synthesis, some of them may be coupled to one another prior to addition to the solid phase reactor. Selection of an appropriate coupling reagent is within the skill of the art. Particularly suitable as a coupling reagent is N,N′-dicyclohexylcarbodiimide (DCC, DIC, HBTU, HATU, TBTU in the presence of HoBt or HoAt).

The activating reagents used in the solid phase synthesis of the peptides are well known in the peptide art. Examples of suitable activating reagents are carbodiimides, such as N,N′-diisopropylcarbodiimide and N-ethyl-N′-(3-dimethylaminopropyl)carbodiimide. Other activating reagents and their use in peptide coupling are described by Schroder & Lubke (1965) and Kapoor (1970).

Each protected amino acid or amino acid sequence is introduced into the solid-phase reactor in about a twofold or more excess, and the coupling may be carried out in a medium of dimethylformamide (DMF):CH₂Cl₂ (1:1) or in DMF or CH₂Cl₂ alone. In cases where intermediate coupling occurs, the coupling procedure is repeated before removal of the α-amino protecting group prior to the coupling of the next amino acid. The success of the coupling reaction at each stage of the synthesis, if performed manually, is preferably monitored by the ninhydrin reaction, as described by Kaiser et al. (1970). Coupling reactions can be performed automatically, as on a Beckman 990 automatic synthesizer, using a program such as that reported in Rivier et al. (1978).

After the desired amino acid sequence has been completed, the intermediate peptide can be removed from the resin support by treatment with a reagent, such as liquid hydrogen fluoride or TFA (if using Fmoc chemistry), which not only cleaves the peptide from the resin but also cleaves all remaining side chain protecting groups and also the α-amino protecting group at the N-terminus if it was not previously removed to obtain the peptide in the form of the free acid. If Met is present in the sequence, the Boc protecting group is preferably first removed using trifluoroacetic acid (TFA)/ethanedithiol prior to cleaving the peptide from the resin with HF to eliminate potential S-alkylation. When using hydrogen fluoride or TFA for cleaving, one or more scavengers such as anisole, cresol, dimethyl sulfide and methylethyl sulfide are included in the reaction vessel.

Cyclization of the linear peptide is preferably affected, as opposed to cyclizing the peptide while a part of the peptido-resin, to create bonds between Cys residues. To effect such a disulfide cyclizing linkage, fully protected peptide can be cleaved from a hydroxymethylated resin or a chloromethylated resin support by ammonolysis, as is well known in the art, to yield the fully protected amide intermediate, which is thereafter suitably cyclized and deprotected. Alternatively, deprotection, as well as cleavage of the peptide from the above resins or a benzhydrylamine (BHA) resin or a methylbenzhydrylamine (MBHA), can take place at 0° C. with hydrofluoric acid (HF) or TFA, followed by oxidation as described above.

The peptides are also synthesized using an automatic synthesizer. Amino acids are sequentially coupled to an MBHA Rink resin (typically 100 mg of resin) beginning at the C-terminus using an Advanced Chemtech 357 Automatic Peptide Synthesizer. Couplings are carried out using 1,3-diisopropylcarbodimide in N-methylpyrrolidinone (NMP) or by 2-(1H-benzotriazole-1-yl)-1,1,3,3-tetramethyluronium hexafluorophosphate (HBTU) and diethylisopro-pylethylamine (DIEA). The FMOC protecting group is removed by treatment with a 20% solution of piperidine in dimethylformamide(DMF). Resins are subsequently washed with DMF (twice), followed by methanol and NMP.

The compounds described herein are used as neurmuscular blocking agents in conjunction with surgery or for intubation of the trachea by conventional parenteral administration e.g., intramuscular or intravenous administration in solution. Thus, the present invention relates to a method for treating a patient during surgical procedures requiring anesthesia and musculoskeletal relaxation. In particular, the method comprises administering to the patient an amount of a compound effective for providing relaxation of muscle. The method involves administering an effective amount of a compound selected from the general formulae which are set forth hereinbefore. The present invention relates to a pharmaceutical composition incorporating a compound described herein or its pharmaceutically acceptable salts.

The manner in which the compounds are administered can vary. Although it is possible to administer the compound in the form of a bulk active chemical, it is preferred to present the compound in the form of a pharmaceutical composition or formulation for parenteral administration. Pharmaceutical compositions containing a compound of the present invention as the active ingredient can be prepared according to conventional pharmaceutical compounding techniques. See, for example, Remington's Pharmaceutical Sciences, 18th Ed. (1990, Mack Publishing Co., Easton, Pa.). Typically, an amount of active ingredient effective to provide muscle relaxation will be admixed with a pharmaceutically acceptable carrier.

The pharmaceutical composition also can include various other components as additives or adjuncts. Exemplary pharmaceutically acceptable components or adjuncts include anesthetics, preservatives, antioxidants, bacteriostatic agents, buffering agents, analgesics, anti-inflammatory agents, anti-pyretics, stabilizing agents, thickening agents and suspending agents. Such components can provide additional therapeutic benefit, or act towards preventing any potential side effects which may be posed as a result of administration of the pharmaceutical composition.

Typically, the pharmaceutical composition is administered as an aqueous or non-aqueous solution, as a suspension, or as an emulsion in a pharmaceutically acceptable liquid or mixture of liquids. The compound within the pharmaceutical composition is administered internally by injection or intravenously. For example, the pharmaceutical composition can be administered intravenously as an infusion (e.g., within aqueous dextrose or saline solutions).

Exemplary methods for administering such muscle relaxant compounds (e.g., so as to achieve sterile or aseptic conditions) will be apparent to the skilled artisan. Certain methods suitable for administering compounds useful according to the present invention are set forth in Goodman and Gilman's The Pharmacological Basis of Therapeutics, 7th Ed. (1985). The administration to the patient can be intermittent; or at a gradual, continuous, constant or controlled rate. Administration can be to a warm-blooded animal (e.g. a mammal, such as a mouse, rat, cat, rabbit, dog, pig, cow or monkey); but advantageously is administered to a human being. Administration occurs after general anesthesia is administered. The frequency of administration normally is determined by an anesthesiologist, and typically varies from patient to patient.

The dose of the compound is that amount effective to provide a desired effect for a desired time frame. By “effective amount” or “effective dose” is meant that amount parenterally administered (e.g., injected intravenously) sufficient to bind to relevant receptor sites on the musculoskeletal fiber of the patient, and to elicit neuropharmacological effects (e.g., elicit brief depolarization, thus resulting in effective short duration relaxation of skeletal muscle). Short duration typically ranges from about 5 to about 60 minutes.

An effective amount of the compound administered to a patient provides rapid onset and short-lived muscle relaxation. For adult human patients undergoing short surgical procedures, the effective dose of typical compounds injected intravenously generally is from about 0.001 mg/kg to about 0.8 mg/kg body weight, preferably from about 0.05 mg/kg to about 0.5 mg/kg, and more preferably from about 0.05 mg/kg to about 0.3 mg/kg. Following administration of typical compounds in such a concentration range, the onset of paralysis normally develops within 1 to 2 minutes, and is reversible (i.e., muscle tone returns within a short period of time). The compounds of this invention would normally be readministered every 15 to 30 minutes after initial administration or given as a slow continuous infusion depending upon the length of time a muscular block is desired, and as determined by the anesthetist and surgeon in charge of the patient. For adult human patients undergoing long surgical procedures, the effective dose of typical compounds is administered through continuous or intermittent intravenous perfusion at a rate from about 0.001 mg/min to about 0.8 mg/min, preferably from about 0.01 mg/min to about 0.5 mg/min, and more preferably from about 0.01 to about 0.25 mg/min. Following administration of typical compounds in the specified amounts, the onset of paralysis typically develops within 1 to 2 minutes and persists for the duration of the superfusion.

For human patients in the pediatric population undergoing short surgical procedures, the effective dose of typical compounds injected intravenously generally is from about 0.001 mg/kg to about 0.5 mg/kg body weight, preferably from about 0.01 mg/kg to about 0.4 mg/kg, and more preferably from about 0.01 mg/kg to about 0.25 mg/kg. Following administration of typical compounds in such a concentration range, the onset of paralysis normally develops within 1 to 2 minutes, and persists for a short period of time before recovery is achieved. For infants and children undergoing long surgical procedures, the effective dose of typical compounds is administered through continuous or intermittent intravenous perfusion at a rate from about 0.001 mg/min to aobut 0.5 mg/min, preferably from about 0.005 mg/min to about 0.3 mg/min, and more preferably from about 0.005 mg/min to about 0.2 mg/min. The total amount of drug administered using such a parenteral route of administration generally does not exceed a total of 10 mg, often does not exceed 5 mg and frequently does not exceed 2 mg. Following administration of typical compounds in the specified amounts, the onset of paralysis typically develops within 1 to 2 minutes and persists for the duration of the superfusion.

Such formulations are normally presented in unit dosage forms such as ampoules or disposable injection devices, or in multidose forms such as a bottle from which the appropriate dose may be withdrawn. All such formulations should be rendered sterile.

The compounds of this invention may be presented as a powder e.g., as a unit dose in a sealed vial to which sterile water may be added by a needle, e.g., through a seal thereof (such as rubber). A suitable unit dose to obtain a neuromuscular block for mammals is about 1 mg to 100 mg and most preferably 3 to 50 mg. Thus a suitable pharmaceutical parenteral preparation will preferably contain 20 to 100 mg of the compounds described herein in solution. A pharmaceutical formulation may conventional contain 5 to 400 mg, or 10 to 400 mg, and most preferably 5 to 200 mg of the compounds of this invention. A simple and preferred formulation is a solution of a compound described herein in water which may be prepared by simply dissolving the compound into previously sterilized pure, i.e., pyrogen free water under aseptic conditions and sterilizing the solution. The compounds described herein may also be administered as an infusion of a dextrose solution or a saline solution e.g., Ringers' Solution.

### EXAMPLES

The present invention is described by reference to the following Examples, which are offered by way of illustration and are not intended to limit the invention in any manner. Standard techniques well known in the art or the techniques specifically described below were utilized.

### Example 1

### Dose-Effect Study for MI and GI

This study was an open label, dose-ranging, single center investigation. A total of 14 rats were studied (10 in each of five groups). All animals were anesthetized with pentobarbital (60 mg/kg) given by intraperitoneal administration and maintained with supplemental doses as determined by physiological monitoring variables. A tracheotomy was performed and the rats were ventilated with room air keeping P_{CO2} near 35 torr. The carotid artery was cannulated to measure blood pressure and arterial blood gases. The right jugular vein was cannulated for intravenous infusion and further drug administration. Body temperature was maintained at 36°-38° C. during the entire experiment. The sciatic nerve was exposed in the popliteal space and stimulated with train-of-four stimulation using a Digistim nerve stimulator. The tivialis anterior muscle contractoin was measured by attaching the rat hind limb to an isometric force transducer to record the evoked response. Prior to administration of the study drug, baseline measurements of blood pressure, heart rate and muscle contraction force were measured for a five-minute period and at five minute intervals for the duration of the study.

The initial dose for analysis was based on biologically effective doses determined in mice. Based on the onset, maximum effect and duration of effect from the first animal studied, the dose for the next animal was either doubled or halved. If the relaxation level was maintained at a maximal level for greater than 20 minutes from this initial dose, then the subsequent dose studied was doubled. this progression continued until the dose that produced near maximal muscle relaxation was found.

The conopeptide derivatives MI and GI were studied in the initial study. For each compound studied, the onset of muacle relaxation, duration of relaxation and an estimate of the ED₅₀ was determined from evoked force transducer response. Onset of relaxation is defined as the time for the evoked response to diminish to 5% of pre-drug baseline. In addition, clinical duration, defined as the time from the administration of drug until the evoked muscle response returns to 25% of its pre-drug baseline, and recovery time, defined as tghe time until evoked response returns to 75% of baseline, were also determined. Data were summarized for each compound.

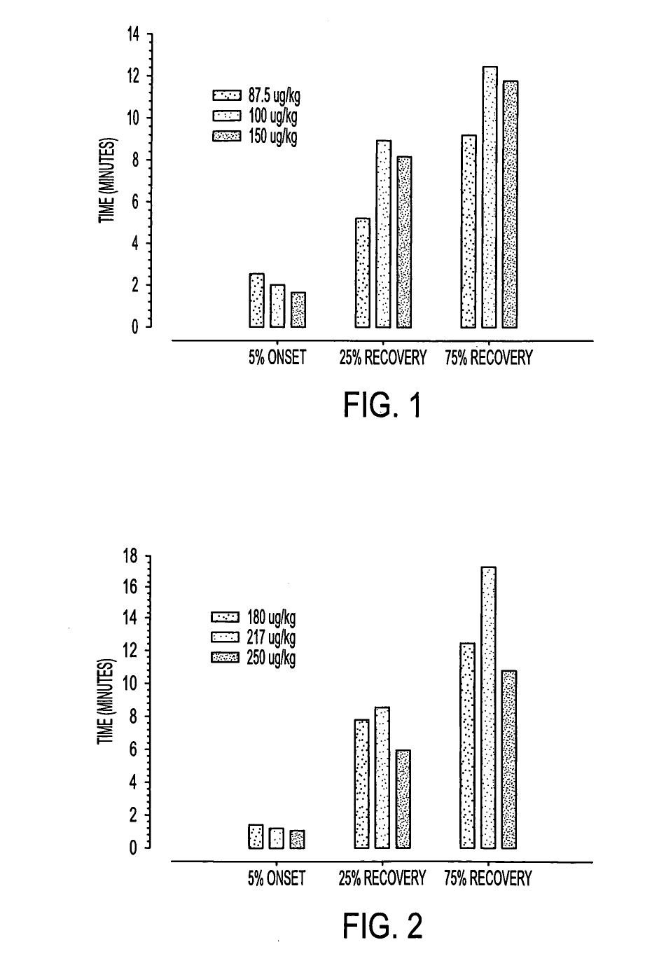

The onset and recovery results for both MI and GI are shown in FIGS. 1 and 2, respectively. MI had a shortest onset of 1.46 minutes. The onset time increased with decreasting dose size as is typical for may neuromuscular blocking agents. The recovery time to 25% and 75% of baseline occurred in approximately 8 and 12 minutes, respectively. These recovery times were constant for doses over 100 μg/kg, which implies that recovery of thge drug effects is very rapid and not easily saturated in its capacity. Anesthetic drugs that behave in similar fashion tend to be degraded by chemical or enzymatic processes in the body rather than by metabolic organ transformation.

GI had a shorter onset time of just under 1 minute. The time for 25% and 75% recovery of baseline was in the range of 8 and 15 minutes, respectively. As with MI, increasing the dose tended to shorten the onset time without extending the recovery times dramatically. For GI, the onset time was similar to that seen with succinylcholine. The recovery times for both agents were similar to succinylcholine.

A comparison of these results to onset and recovery times for other clinically available neuromuscular blocking agents is shown in Table 1.

TABLE 1
Comparison of Neuromuscular Blocking Agents

Recovery (min)

| | | | |
|---|---|---|---|
| Agent (mg/kg) | Onset Time (sec) | 25% | 75% |

| | | | |
|---|---|---|---|
| MI (0.15) | 90 | 8 | 12 |
| GI (0.2) | 60-70 | 6-8 | 10-15 |
| Sux (1.0) | 60 | 5-7 | 10 |
| Org 9847 (1.5) | 80 | 8 | 15 |
| Rocuronium (0.6) | 80 | 40 | 60 |
| Mivacurium (0.2) | 150 | 20 | 27 |
| Vecuronium (0.1) | 120-180 | 40 | 60 |
| Cisatracurium (0.1) | 120-180 | 45 | 60-70 |

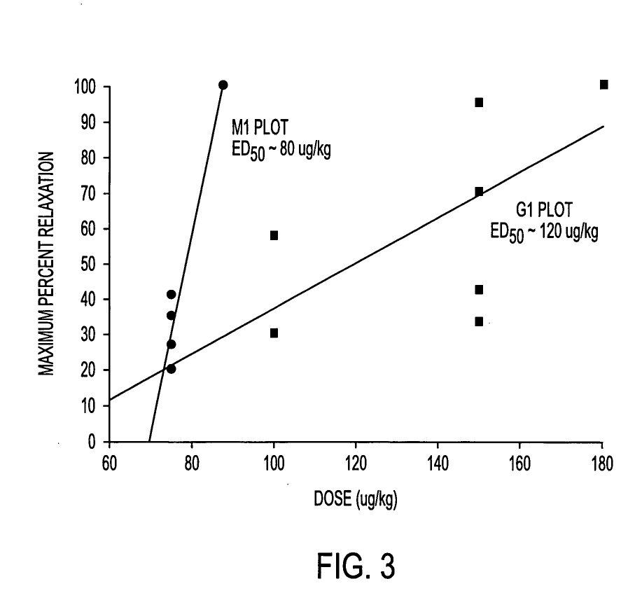

For doses of these agents which produced less than maximum levels of neuromuscular block, dose-response plots can be determined to estimate the ED₅₀ dose of these agents. In this context, ED₅₀ refers to the dose of agent which is expected to produce half of the maximum relaxation level. The data of this initial study (FIG. 3) shows that GI is less potent than MI as reflected in the lower ED₅₀ value for MI (˜80 μg/kg for MI compared to ˜120 μg/kg for GI).

These results show that α-conotoxin peptides are biologically active at the neuromuscular junction producing skeletal muscle paralysis that mimics the repsonse seen with non-depolarizing neuromuscular blocking agents given during anesthesia. The onset and duration of relaxation is rapid and short which is highly desirable for a number of clinical reasons. In this regard, with the rapid onset time, short duration and no prolongation of drug effect with large doses, the clinical benefit of the α-conotoxin peptides exceeds the currently available non-depolarizing neuromuscular blocking agents. In addition to their desirable effect profile, the α-conotoxin peptides appear to have no significant cardiovascular effects on administration. Thus, the desirable effect profile with minimal side effects are desirable clinical properties for the α-conotoxin peptides.

### Example 2

### Dose-Effect Study for Iodinated-MI

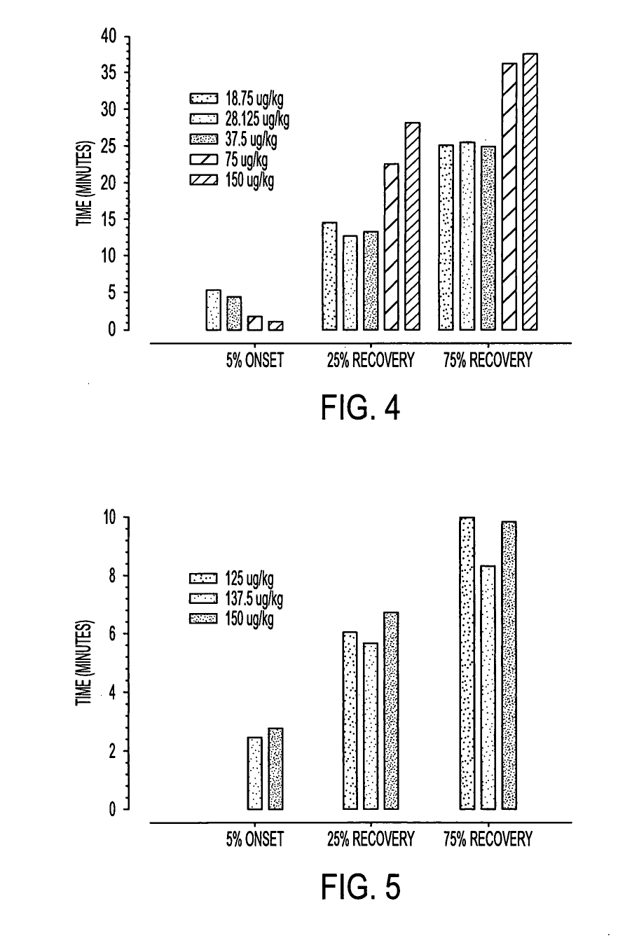

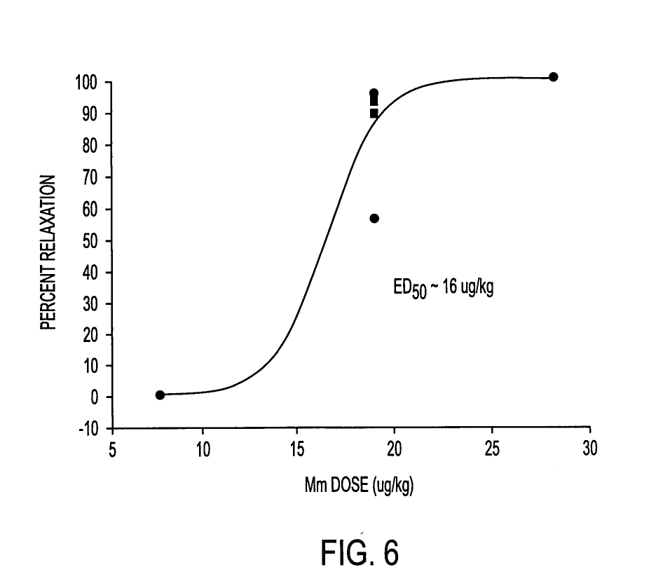

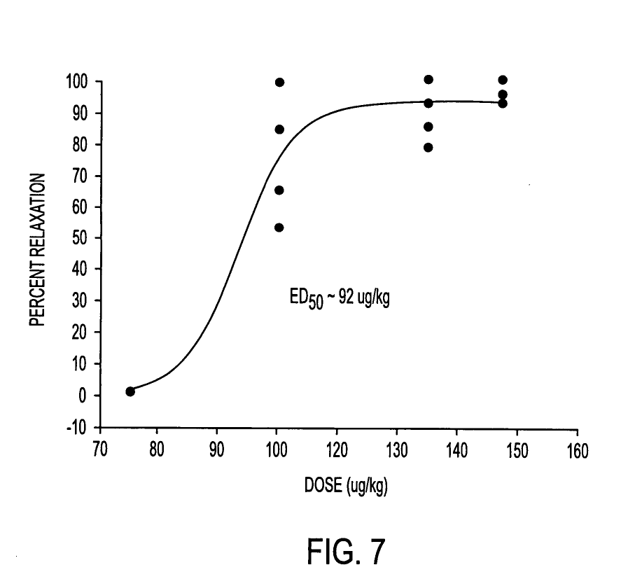

A similar study as described in Example 1 was conducted for two iodinated derivatives of MI, namely, mono-iodo-Tyr₁₂-MI and di-iodo-Tyr₁₂-MI. The onset and recovery results for mono-iodo-Tyr₁₂-MI and di-iodo-Tyr₁₂-MI are shown in FIGS. 4 and 5, respectively. Dose-response plots for mono-iodo-Tyr₁₂-MI and di-iodo-Tyr₁₂-MI were made to estimate the ED₅₀ dose of these agents. The ED₅₀ values are ˜16 g/kg formono-iodo-Tyr₁₂-MI and ˜92.5 μg/kg for di-iodo-Tyr₁₂-MI.

### Example 3

### Muscle Relaxant Effect in Anesthetized Monkeys

The peptides MI, GI, EI, mono-iodo-MI and di-iodo-MI are each separately dissolved 0.9 percent saline at a concentration of 2 mg/ml. Rhesus monkeys are anesthetized with halothane, nitrous oxide and oxygen. The maintenance concentration of halothane is 1.0%. Arterial and venous catheters are placed in the femoral vessels for drug administration and recording of the arterial pressure. Controlled ventilation is accomplished via an endotrachael tube. Twitch and tetanic contractions of the tibialis arterior muscle are elicited indirectly via the sciatic nerve. Recordings of arterial pressure electrocardiogram (lead I), heart rate, and muscle function are made simultaneously. Four to six animals received each listed compound. Four additional animals received succinylcholine chloride or d-tubocurarine chloride as controls. Is is seen that the tested compounds generally provide similar or better results than those seen for succinylcholine chloride or d-tubocurarine chloride.

### Example 4

### Isolation of DNA Encoding α-Conotoxins

DNA coding for α-conotoxins was isolated and cloned in accordance with conventional techniques using general procedures well known in the art, such as described in Olivera et al. (1996). Alternatively, cDNA libraries was prepared from Conus venom duct using conventional techniques. DNA from single clones was amplified by conventional techniques using primers which correspond approximately to the M13 universal priming site and the M13 reverse universal priming site. Clones having a size of approximately 300 nucleotides were sequenced and screened for similarity in sequence to known α-conotoxins. The DNA sequences and encoded propeptide or peptide sequences are set forth in Tables 2-38. It was discovered that the following mature α-conotoxin peptides had the same sequence: (a) R1.4, A1.1, Bt1.6, Cn1.1 and MnI; and (b) Sm1.1 and Cr1.1.

TABLE 2

DNA Sequence (SEQ ID NO:28) and Protein Sequence (SEQ ID NO:29)
of GI

atg ttc acc gtg ttt ctg ttg gtg gtc ttg gca acc act gtc gtt tcc
Met Phe Thr Val Phe Leu Leu Val Val Leu Ala Thr Thr Val Val Ser
ttc cct tca gaa cgt gca tct gat ggc agg gat gac aca gcc aaa gac
Phe Pro Ser Glu Arg Ala Ser Asp Gly Arg Asp Asp Thr Ala Lys Asp
gaa ggg tct gac atg gag aaa ttg gtc gag aaa aaa gaa tgt tgc aat
Glu Gly Ser Asp Met Glu Lys Leu Val Glu Lys Lys Glu Cys Cys Asn
cct gcc tgt ggc aga cac tac agt tgt gga cgc tgatgctcca ggaccctctg
Pro Ala Cys Gly Arg His Tyr Ser Cys Gly Arg
aaccacggac gtgccgccct ctgcctgacc tgcttcactg tccgtctctt tgtgccacta
gaactgaaca gctcgatcca ctagactacc acgttacctc cgtgttctaa aactacttgg
tttagattgc ctttaatttc tagtcatact tcctgttatt acgtcgtcca aaattgaaac
aagaacatga ggggtgtcag ctcaaacaaa atcaggcaat gacaaggaaa atgtctccga
tcgatccgaa aactgtcacc cgtcactctc ttaaccagtt ttagaactga ttaccactag
agcttttgta ccacatcaaa tcaggtctat gtgtgatgtt tcttttgcaa aatttaattt
ttgagaaaaa aagctcaaaa tgtgggaagt gcttttgatt ttctgacaac ttgtgatcat
gtccgttttc agtgagtcta attgcaacct ctgtgtgatt ttcttcacct gttaagcaac
gcaaagaggt tgtccataac caggaaagca acagacaaag aaatgcttga gaatttcagg
ttatagataa ggtaaggaaa aaaaggagag ctatgggaaa tgatgaaaac aacagataaa
ataaattgaa cagtacctac ttgtttcatg gttgattttt ttttctctga ataatctctg
tggacactaa tggcagtctc tcctcacccc acgccattag taagcttatt ttttctttct
ttatccaaga tttgctgaac atatttagcc tagatataga cattgctaca tatataatct
gacaataaac tttcatgggc accaatt

TABLE 3

DNA Sequence (SEQ ID NO:30) and Protein Sequence (SEQ ID NO:31)
of SIB

atg ttc acc gtg ttt ctg ttg gtt gtc ttg gca acc act gtc gtt tcc
Met Phe Thr Val Phe Leu Leu Val Val Leu Ala Thr Thr Val Val Ser
ttc cct tca gat cgt gca tct gat ggc agg gat gac gaa gcc aaa gac
Phe Pro Ser Asp Arg Ala Ser Asp Gly Arg Asp Asp Glu Ala Lys Asp
gaa agg tct gac atg cac gaa tcg gac cgg aaa gaa atc tgt tgc aat
Glu Arg Ser Asp Met His Glu Ser Asp Arg Lys Glu Ile Cys Cys Asn
cct gcc tgt ggc cca aag tat agt tgt gga cgc tgatgctcca ggaccctctg
Pro Ala Cys Gly Pro Lys Tyr Ser Cys Gly Arg
aacc

TABLE 4

DNA Sequence (SEQ ID NO:32) and Protein Sequence (SEQ ID NO:33)
of R1

atg ttc acc gtg ttt ctg ttg gtt gtc ttg aca atc act gtc gtt tcc
Met Phe Thr Val Phe Leu Leu Val Val Leu Thr Ile Thr Val Val Ser
ttc cct tca gat cgt gca tct gat ggc agg gat gac gaa gcc aaa gac
Phe Pro Ser Asp Arg Ala Ser Asp Gly Arg Asp Asp Glu Ala Lys Asp
gaa agg tct gac atg tac aaa tcg aaa cgg aat gga cgc tgt tgc cat
Glu Arg Ser Asp Met Tyr Lys Ser Lys Arg Asn Gly Arg Cys Cys His
cct gcc tgt ggc aaa cac ttt agt tgt gga cgc tgatgctcca ggaccctctg
Pro Ala Cys Gly Lys His Phe Ser Cys Gly Arg
aaccacgacg t

TABLE 5

DNA Sequence (SEQ ID NO:34) and Protein Sequence (SEQ ID NO:35)
of R1.3

atg ttc acc gtg ttt ctg ttg gtg gtc ttg gca acc act gtc gtt tcc
Met Phe Thr Val Phe Leu Leu Val Val Leu Ala Thr Thr Val Val Ser
ttc cct tca gaa cgt gca tct gat ggc agg gat gac aca gcc aaa gac
Phe Pro Ser Glu Arg Ala Ser Asp Gly Arg Asp Asp Thr Ala Lys Asp
gaa ggg tct gac atg gag aaa ttg gtc gag aaa aaa gaa tgt tgc aat
Glu Gly Ser Asp Met Glu Lys Leu Val Glu Lys Lys Glu Cys Cys Asn
cct gcc tgt ggc aga cac tac agt tgt aag gga ggacgctgat gctccagacc
Pro Ala Cys Gly Arg His Tyr Ser Cys Lys Gly
ctctgaacca cgacgt

TABLE 6

DNA Sequence (SEQ ID NO:36) and Protein Sequence (SEQ ID NO:37)
of R1.4

atg ttc acc gtg ttt ctg ttg gtt gtc ttg aca atc act gtc gtt tcc
Met Phe Thr Val Phe Leu Leu Val Val Leu Thr Ile Thr Val Val Ser
ttc cct tca gat cgt gca tct gat ggc agg gat gac gaa gcc aaa gac
Phe Pro Ser Asp Arg Ala Ser Asp Gly Arg Asp Asp Glu Ala Lys Asp
gaa agg tct gac atg tac aaa tcg aaa cgg aat gga cgc tgt tgc cat
Glu Arg Ser Asp Met Tyr Lys Ser Lys Arg Asn Gly Arg Cys Cys His
cct gcc tgt ggc aaa cac ttt agt tgt gga cgc tgatgctcca ggaccctctg
Pro Ala Cys Gly Lys His Phe Ser Cys Gly Arg
aaccacgacg t

TABLE 7

DNA Sequence (SEQ ID NO:28) and Protein Sequence (SEQ ID NO:39)
of Sm1.1

atg ttc acc gtg ttt ctg ttg gtt gtc ttg gca acc act gtc gtt tcc
Met Phe Thr Val Phe Leu Leu Val Val Leu Ala Thr Thr Val Val Ser
ttc cct tca gat cgt gca tct gat ggc agg gat gac gaa gcc aaa gac
Phe Pro Ser Asp Arg Ala Ser Asp Gly Arg Asp Asp Glu Ala Lys Asp
gaa agg tct gac atg cac gaa tcg ggc cgg aaa gga cgc gga cgc tgt
Glu Arg Ser Asp Met His Glu Ser Gly Arg Lys Gly Arg Gly Arg Cys
tgc cat cct gcc tgt ggc cca aac tat agt tgt ggacgctgat gctccaggac
Cys His Pro Ala Cys Gly Pro Asn Tyr Ser Cys
cctctgaacc acgacgt

TABLE 8

DNA Sequence (SEQ ID NO:40) and Protein Sequence (SEQ ID NO:41)
of SIIA

atg ttc acc gtg ttt ctg ttg gtt gtc ttg gca acc act gtc gtt tcc
Met Phe Thr Val Phe Leu Leu Val Val Leu Ala Thr Thr Val Val Ser
ttc cct tca gat cgt gca tct gat ggc agg gat gac gaa gcc aaa gac
Phe Pro Ser Asp Arg Ala Ser Asp Gly Arg Asp Asp Glu Ala Lys Asp
gaa agg tct gac atg cac gaa tcg gac cgg aat gga cgc gga tgc tgt
Glu Arg Ser Asp Met His Glu Ser Asp Arg Asn Gly Arg Gly Cys Cys
tgc aat cct gcc tgt ggc cca aac tat ggt tgt ggc acc tca tgc tcc
Cys Asn Pro Ala Cys Gly Pro Asn Tyr Gly Cys Gly Thr Ser Cys Ser
agg acc ctc tgaaccacga cgttcgagca
Arg Thr Leu

TABLE 9

DNA Sequence (SEQ ID NO:42) and Protein Sequence
(SEQ ID NO:43) of S11

tgt tgc cat cct gcc tgt ggc aga aag tat aat tgt gga cgc tga
Cys Cys His Pro Ala Cys Gly Arg Lys Tyr Asn Cys Gly Arg

TABLE 10

DNA Sequence (SEQ ID NO:44) and Protein Sequence (SEQ ID NO:45)
of S2

tgc tgt tgc aat cct gcc tgt ggc cca aac tat ggt tgt ggc acc tca
Cys Cys Cys Asn Pro Ala Cys Gly Pro Asn Tyr Gly Cys Gly Thr Ser
tgc tcc aga ccc tct gaa cca cga cgt tag
Cys Ser Arg Pro Ser Glu Pro Arg Arg

TABLE 11

DNA Sequence (SEQ ID NO:46) and Protein Sequence (SEQ ID NO:47)
of GIB

atg ttc acc gtg ttt ctg ttg gtg gtc ttg gca acc act gtc gtt tcc
Met Phe Thr Val Phe Leu Leu Val Val Leu Ala Thr Thr Val Val Ser
ttc cct tca gaa cgt gca tct gat ggc agg gat gac aca gcc aaa gac
Phe Pro Ser Glu Arg Ala Ser Asp Gly Arg Asp Asp Thr Ala Lys Asp
gaa ggg tct gac atg gag aaa ttg gtc gag aaa aaa gaa tgt tgc aat
Glu Gly Ser Asp Met Glu Lys Leu Val Glu Lys Lys Glu Cys Cys Asn
cct gcc tgt ggc aga cac tac agt tgt aag gga ggacgctgat gctccaggac
Pro Ala Cys Gly Arg His Tyr Ser Cys Lys Gly
cctctgaacc acggacgtgc cgccctctgc ctgacctgct tcactgtccg tctctttgtg
ccactagaac tgaacagctc gatccactag actaccacgt tacctccgtg ttctaaaact
acttggttta gattgccttt aatttctagt catacttcct gttattacgt cgtccaaaat
tgaaacaaga acatgagggg tgtcagctca aacaaaatca ggcaatgaca aggaaaatgt
ctccgatcga tccgaaaact gtcacccgtc actctcttaa ccagttttag aactgattac
cactagagct tttgtaccac atcaaatcag gtctatgtgt gatgtttctt ttgcaaaatt
taatttttga gaaaaaaagc tcaaaatgtg ggaagtgctt ttgattttct gacaacttgt
gatcatgtcc gttttcagtg agtctaattg caacctctgt gtgattttct tcacctgtta
agcaacgcaa agaggttgtc cataaccagg aaagcaacag acaaagaaat gcttgagaat
ttcaggttat agataaggta aggaaaaaaa ggagagctat gggaaatgat gaaaacaaca
gataaaataa attgaacagt acctacttgt ttcatggttg attttttttt ctctgaataa
tctctgtgga cactaatggc agtctctcct caccccacgc cattagtaag cttatttttt
ctttctttat ccaagatttg ctgaacatat ttagcctaga tatagacatt gctacatata
taatctgaca ataaactttc atgggcacca att

TABLE 12

DNA Sequence (SEQ ID NO:48) and Protein Sequence (SEQ ID NO:49)
of MnII

atg ttc acc gtg ttt ctg ttg gtt gtc ttg aca acc act gtc gtt tcc
Met Phe Thr Val Phe Leu Leu Val Val Leu Thr Thr Thr Val Val Ser
ttc cct tca gat agt gca tct ggt ggc agg gat gac gag gcc aaa gac
Phe Pro Ser Asp Ser Ala Ser Gly Gly Arg Asp Asp Glu Ala Lys Asp
gaa agg tct gac atg tac gaa ttg aaa cgg aat gga cac tgt tgc cat
Glu Arg Ser Asp Met Tyr Glu Leu Lys Arg Asn Gly His Cys Cys His
cct gcc tgt ggt ggc aaa tac gtt aaa tgt gga cgc tgatgctcca
Pro Ala Cys Gly Gly Lys Tyr Val Lys Cys Gly Arg
ggaccctctc gaaccacg

TABLE 13

DNA Sequence (SEQ ID NO:50) and Protein Sequence (SEQ ID NO:51)
of A1.2

atg ttc acc gtg ttt ctg ttg gtt gtc ttg aca acc act gtc gtt tcc
Met Phe Thr Val Phe Leu Leu Val Val Leu Thr Thr Thr Val Val Ser
ttc cct tca gat agt gca tct ggt ggc agg gat gac gag gcc aaa gac
Phe Pro Ser Asp Ser Ala Ser Gly Gly Arg Asp Asp Glu Ala Lys Asp
gaa agg tct gac atg tac gaa ttg aaa cgg aat gga cgc tgt tgc cat
Glu Arg Ser Asp Met Tyr Glu Leu Lys Arg Asn Gly Arg Cys Cys His
cct gcc tgt ggt ggc aaa tac gtt aaa tgt gga cgc tgatgctcca
Pro Ala Cys Gly Gly Lys Tyr Val Lys Cys Gly Arg
ggaccctctc gaaccacg

TABLE 14

DNA Sequence (SEQ ID NO:52) and Protein Sequence (SEQ ID NO:53)
of A1.1

atg ttc acc gtg ttt ctg ttg gtt gtc ttg aca aca act gtc gtt tcc
Met Phe Thr Val Phe Leu Leu Val Val Leu Thr Thr Thr Val Val Ser
tac cct tca gat agt gca tct gat ggc agg gat gac gaa gcc aaa gac
Tyr Pro Ser Asp Ser Ala Ser Asp Gly Arg Asp Asp Glu Ala Lys Asp
gaa agg tct gac atg tac aaa tcg aaa cgg aat gga cgc tgt tgc cat
Glu Arg Ser Asp Met Tyr Lys Ser Lys Arg Asn Gly Arg Cys Cys His
cct gcc tgt ggc aaa cac ttt agt tgt gga cgc tgatgctcca ggaccctctg
Pro Ala Cys Gly Lys His Phe Ser Cys Gly Arg
aaccacgacg t

TABLE 15

DNA Sequence (SEQ ID NO:54) and Protein Sequence (SEQ ID NO:55)
of Bt1.6

atg ttc acc gtg ttt ctg ttg gtt gtc ttg gca acc act gtc gtt tcc
Met Phe Thr Val Phe Leu Leu Val Val Leu Ala Thr Thr Val Val Ser
tac cct tca gat agt gca tct gat ggc agg gat gac gaa acc aaa gac
Tyr Pro Ser Asp Ser Ala Ser Asp Gly Arg Asp Asp Glu Thr Lys Asp
gaa aag tct gac atg tac aaa tcg aaa cgg aat gga cgc tgt tgc cat
Glu Lys Ser Asp Met Tyr Lys Ser Lys Arg Asn Gly Arg Cys Cys His
cct gcc tgt ggc aaa cac ttt agt tgt gga cgc tgatgctgca ggaccctctg
Pro Ala Cys Gly Lys His Phe Ser Cys Gly Arg
aaccacgacg t

TABLE 16

DNA Sequence (SEQ ID NO:56) and Protein Sequence (SEQ ID NO:57)
of Cn1.1

atg ttc acc gtg ttt ctg ttg gtt gtc ttg aca acc act gtc gtt tcc
Met Phe Thr Val Phe Leu Leu Val Val Leu Thr Thr Thr Val Val Ser
ttc cct tca gat agt gca tct gat gtc agg gat gac gaa gcc aaa gac
Phe Pro Ser Asp Ser Ala Ser Asp Val Arg Asp Asp Glu Ala Lys Asp
gaa agg tct gac atg tac aaa tcg aaa cgg aat gga cgc tgt tgc cat
Glu Arg Ser Asp Met Tyr Lys Ser Lys Arg Asn Gly Arg Cys Cys His
cct gcc tgt ggc aaa cac ttt agt tgt gga cgc tgatgctcca ggaccctctg
Pro Ala Cys Gly Lys His Phe Ser Cys Gly Arg
aaccacgacg t

TABLE 17

DNA Sequence (SEQ ID NO:58) and Protein Sequence (SEQ ID NO:59)
of MnI

atg ttc acc gtg ttt ctg ttg gtt gtc ttg aca aca act gtc gtt tcc
Met Phe Thr Val Phe Leu Leu Val Val Leu Thr Thr Thr Val Val Ser
tac cct tca gat agt gca tct gat ggc agg gat gac gaa gcc aaa gac
Tyr Pro Ser Asp Ser Ala Ser Asp Gly Arg Asp Asp Glu Ala Lys Asp
gaa agg tct gac atg tac aaa tcg aaa cgg aat gga cgc tgt tgc cat
Glu Arg Ser Asp Met Tyr Lys Ser Lys Arg Asn Gly Arg Cys Cys His
cct gcc tgt ggc aaa cac ttt agt tgt gga cgc tgatgctcca ggaccctctg
Pro Ala Cys Gly Lys His Phe Ser Cys Gly Arg
aaccacgacg t

TABLE 18

DNA Sequence (SEQ ID NO:60) and Protein Sequence (SEQ ID NO:61)
of Cr1.1

atg ttc acc gtg ttt ctg ttg gtt gtc ttg gca gcc act gtc gtt tcc
Met Phe Thr Val Phe Leu Leu Val Val Leu Ala Ala Thr Val Val Ser
ttc cct tca gat cgt gca tct gat ggc agg gat gac gaa gcc aaa gac
Phe Pro Ser Asp Arg Ala Ser Asp Gly Arg Asp Asp Glu Ala Lys Asp
gaa aga tct gac atg cac gaa tcg gac cgg aaa gga cgc gga cgc tgt
Glu Arg Ser Asp Met His Glu Ser Asp Arg Lys Gly Arg Gly Arg Cys
tgc cat cct gcc tgt ggc cca aat tat agt tgt gga cgc tgatgctcca
Cys His Pro Ala Cys Gly Pro Asn Tyr Ser Cys Gly Arg
ggaccctctg aaccacgacg

TABLE 19

DNA Sequence (SEQ ID NO:62) and Protein Sequence (SEQ ID NO:63)
of R1.2

atg ttc acc gtg ttt ctg ttg gtg gtc ttg gca acc act gtc gtt tcc
Met Phe Thr Val Phe Leu Leu Val Val Leu Ala Thr Thr Val Val Ser
ttc cct tca gaa cgt gca tct gat ggc agg gat gac aca gcc aaa gac
Phe Pro Ser Glu Arg Ala Ser Asp Gly Arg Asp Asp Thr Ala Lys Asp
gaa ggg tct gac atg gac aaa ttg gtc gag aaa aaa gaa tgt tgc cat
Glu Gly Ser Asp Met Asp Lys Leu Val Glu Lys Lys Glu Cys Cys His
cct gcc tgt ggc aaa cac ttc agt tgt gga cgc tgatgctcca ggaccctctg
Pro Ala Cys Gly Lys His Phe Ser Cys Gly Arg
aaccacgacg t

TABLE 20

DNA Sequence (SEQ ID NO:64) and Protein Sequence (SEQ ID NO:65)
of A1.3

tct gat ggc agg gat gac gaa gcc aaa gac gaa agg tct gac atg tac
Ser Asp Gly Arg Asp Asp Glu Ala Lys Asp Glu Arg Ser Asp Met Tyr
aaa tcg aaa cgg aat gga cgc tgt tgc cac cct gcc tgt ggc aaa cac
Lys Ser Lys Arg Asn Gly Arg Cys Cys His Pro Ala Cys Gly Lys His
ttt att tgt gga cgc tga
Phe Ile Cys Gly Arg

TABLE 21

DNA Sequence (SEQ ID NO:66) and Protein Sequence (SEQ ID NO:67)
of A1.7

tct ggt ggc agg gat gac gaa gcc aaa gac gaa agg tct gac atg tac
Ser Gly Gly Arg Asp Asp Glu Ala Lys Asp Glu Arg Ser Asp Met Tyr
gaa tcg gac cgg aat gga cgc tgt tgc cat cct gcc tgt ggc aaa cac
Glu Ser Asp Arg Asn Gly Arg Cys Cys His Pro Ala Cys Gly Lys His
ttt agt tgt gga cgc tga
Phe Ser Cys Gly Arg

TABLE 22

DNA Sequence (SEQ ID NO:68) and Protein Sequence (SEQ ID NO:69)
of A1.8

tct gat ggc agg gat gac gaa gcc aaa gac aaa agg tct gac atg tac
Ser Asp Gly Arg Asp Asp Glu Ala Lys Asp Lys Arg Ser Asp Met Tyr
gaa tcg gac cgg aat gga cgc tgt tgc cat cct tcc tgt ggc aga aag
Glu Ser Asp Arg Asn Gly Arg Cys Cys His Pro Ser Cys Gly Arg Lys
tat aat tgt gga cgc tga
Tyr Asn Cys Gly Arg

TABLE 23

DNA Sequence (SEQ ID NO:70) and Protein Sequence (SEQ ID NO:71)
of Ay1.1

tctgatggca gggatgacga agccaaagac gaaaggtctg acatgtac gaa tcg gac
Glu Ser Asp
cgg aat gga cgc tgt tgc cat cct gcc tgt gcg aga aag tat aat tgt
Arg Asn Gly Arg Cys Cys His Pro Ala Cys Ala Arg Lys Tyr Asn Cys
gga cgc tgatgctcca ggaccctctg aaccacgacg t
Gly Arg

TABLE 24

DNA Sequence (SEQ ID NO:72) and Protein Sequence (SEQ ID NO:73)
of Ay1.1a

tctgatggca gggatgacga agccaaagac gaaaggtctg acatgtac gaa tcg gag
Glu Ser Glu
cgg aat gaa cgc tgt tgc cat cct gcc tgt gcg aga aag tat aat tgt
Arg Asn Glu Arg Cys Cys His Pro Ala Cys Ala Arg Lys Tyr Asn Cys
gga cgc tgatgctcca ggaccctctg aaccacgacg t
Gly Arg

TABLE 25

DNA Sequence (SEQ ID NO:74) and Protein Sequence (SEQ ID NO:75)
of M1.1

atg ttc acc gtg ttt ctg ttg gtt gtc ttg aca acc act gtc gtt tcc
Met Phe Thr Val Phe Leu Leu Val Val Leu Thr Thr Thr Val Val Ser
ttc cct tca gat cgt gca tct gat ggc agg gat gac gaa gcc aaa gac
Phe Pro Ser Asp Arg Ala Ser Asp Gly Arg Asp Asp Glu Ala Lys Asp
gaa agg tct gac atg tac gaa tcg aaa cgg gat gga cgc tgt tgc cat
Glu Arg Ser Asp Met Tyr Glu Ser Lys Arg Asp Gly Arg Cys Cys His
cct gcc tgt ggg caa aac tat agt tgt gga cgc tgatgctcca ggaccctctg
Pro Ala Cys Gly Gln Asn Tyr Ser CYS Gly Arg
aaccacgacg t

TABLE 26

DNA Sequence (SEQ ID NO:76) and Protein Sequence (SEQ ID NO:77)
of M1.3

tct gat ggc agg gat gac gaa gcc aaa gac gaa agg cct gac atg tac
Ser Asp Gly Arg Asp Asp Glu Ala Lys Asp Glu Arg Pro Asp Met Tyr
aaa tcg aaa cgg gat gga cgc tgt tgc cat cct gcc tgt gcg aaa cac
Lys Ser Lys Arg Asp Gly Arg Cys Cys His Pro Ala Cys Ala Lys His
ttt aat tgt gga cgc tgatgctcca ggaccctctg aaccacgacg t
Phe Asn Cys Gly Arg

TABLE 27

DNA Sequence (SEQ ID NO:78) and Protein Sequence (SEQ ID NO:79)
of M1.4

tct gat ggc agg gat gac gaa gcc aaa gac gaa agg tct gac atg tac
Ser Asp Gly Arg Asp Asp Glu Ala Lys Asp Glu Arg Ser Asp Met Tyr
gaa tcg aaa cgg aat gga cgc tgt tgc cat cct gcc tgt gcg aaa aac
Glu Ser Lys Arg Asn Gly Arg Cys Cys His Pro Ala Cys Ala Lys Asn
tat agt tgt gga cgc tgatgctcca ggaccctctg aaccacgacg t
Tyr Ser Cys Gly Arg

TABLE 28

DNA Sequence (SEQ ID NO:80) and Protein Sequence (SEQ ID NO:81)
of M1.5

tct gat ggc agg gat gac gaa gcc aaa gac gaa agg tct gac atg tac
Ser Asp Gly Arg Asp Asp Glu Ala Lys Asp Glu Arg Ser Asp Met Tyr
gaa tcg gac cgg aat gga cgc tgt tgc cat cct gcc tgt gcg aga aag
Glu Ser Asp Arg Asn Gly Arg Cys Cys His Pro Ala Cys Ala Arg Lys
tat aat tgt gga cgc tgatgctcca ggaccctctg aaccacgacg t
Tyr Asn Cys Gly Arg

TABLE 29

DNA Sequence (SEQ ID NO:82) and Protein Sequence (SEQ ID NO:83)
of O1.3

tctgatggca gggatgacac agccaaaaac aaaggatctg acatgaacaa attg gtc
Val
aag aaa aaa caa tgt tgc aat cct gcc tgt ggc cca aag tat agt tgt
Lys Lys Lys Gln Cys Cys Asn Pro Ala Cys Gly Pro Lys Tyr Ser Cys
gga cac tgatgctcca ggaccctctg aaccacgacg t
Gly His

TABLE 30

DNA Sequence (SEQ ID NO:84) and Protein Sequence (SEQ ID NO:85)
of S1.3

tct gat ggc agg gat gac gaa goc aaa gac gaa agg tct gac atg cac
Ser Asp Cly Arg Asp Asp Glu Ala Lys Asp Glu Arg Ser Asp Met His
gaa tcg gac cgg aaa gga cgc gca tac tgt tgc cat cct gcc tgt ggc
Glu Ser Asp Arg Lys Gly Arg Ala Tyr Cys Cys His Pro Ala Cys Gly
aaa aag tat aat tgt gga cgc tgatgctcca ggaccctctg aaccacgacg t
Lys Lys Tyr Asn Cys Gly Arg

TABLE 31

DNA Sequence (SEQ ID NO:86) and Protein Sequence (SEQ ID NO:87)
of EI

atg ttc acc gtg ttt ctg ttg gtt gtc ttg gca acc act gtc ggt tcc
Met Phe Thr Val Phe Leu Leu Val Val Leu Ala Thr Thr Val Gly Ser
ttc act tta gat cgt gca tct gat ggt agg gat gcc gca gcc aac gac
Phe Thr Leu Asp Arg Ala Ser Asp Gly Arg Asp Ala Ala Ala Asn Asp
aaa gcg tct gac ctg atc gct ctg acc gcc agg aga gat cca tgc tgt
Lys Ala Ser Asp Leu Ile Ala Leu Thr Ala Arg Arg Asp Pro Cys Cys
tac cat cct acc tgt aac atg agt aat cca cag att tgt ggt
Tyr His Pro Thr Cys Asn Met Ser Asn Pro Gln Ile Cys Gly
tgaagacgct gatgctccag gaccctctga accacgacgt

TABLE 32

DNA Sequence (SEQ ID NO:88) and Protein Sequence (SEQ ID NO:89)
of EIA

atg ttc acc gtg ttt ctg ttg gtt gtc ttg gca acc act gtc ggt tcc
Met Phe Thr Val Phe Leu Leu Val Val Leu Ala Thr Thr Val Gly Ser
ttc act tta gat cgt gca tct gat ggt agg gat gcc gca gcc aac gac
Phe Thr Leu Asp Arg Ala Ser Asp Gly Arg Asp Ala Ala Ala Asn Asp
aaa gcg tct gac ctg atc gct ctg acc gcc agg aga gat cca tgc tgt
Lys Ala Ser Asp Leu Ile Ala Leu Thr Ala Arg Arg Asp Pro Cys Cys
tcc aat cct gcc tgt aac gtg aat aat cca cag att tgt ggt
Ser Asn Pro Ala Cys Asn Val Asn Asn Pro Gln Ile Cys Gly
tgaagacgct gatgctccag gaccctctga accacgacgt

TABLE 33

DNA Sequence (SEQ ID NO:90) and Protein Sequence (SEQ ID NO:91)
of P1.2

atg ttc acc gtg ttt ctg ttg gtg gat gcc gca gcc aac gac aag gcg
Met Phe Thr Val Phe Leu Leu Val Asp Ala Ala Ala Asn Asp Lys Ala
tct gac cgg atc gct ctg acc gcc agg aga gat cca tgc tgt tcc aat
Ser Asp Arg Ile Ala Leu Thr Ala Arg Arg Asp Pro Cys Cys Ser Asn
cct gtc tgt acc gtg cat aat cca cag att tgt ggt tgaagacgct
Pro Val Cys Thr Val His Asn Pro Gln Ile Cys Gly
gatgctccag gaccctctga accacgacgt

TABLE 34

DNA Sequence (SEQ ID NO:92) and Protein Sequence (SEQ ID NO:93)
of P1.3

atg ttc acc gtg ttt ctg ttg gtt gtc ttg gta acc acc gtc gtt tcc
Met Phe Thr Val Phe Leu Leu Val Val Leu Val Thr Thr Val Val Ser
ttc aat tca gat cgt gca tta ggt ggc agg aat gct gca gcc aaa gcg
Phe Asn Ser Asp Arg Ala Leu Gly Gly Arg Asn Ala Ala Ala Lys Ala
tct gac aag atc gct tcg atc ctc ggg aga aga gca tgc tgt tct tat
Ser Asp Lys Ile Ala Ser Ile Leu Gly Arg Arg Ala Cys Cys Ser Tyr
cct ccc tgt aac gtg aac tat cca gaa att tgt ggt gga cga ggc
Pro Pro Cys Asn Val Asn Tyr Pro Glu Ile Cys Gly Gly Arg Gly
tgatgctcca ggaccctctg aaccacgacg t

TABLE 35

DNA Sequence (SEQ ID NO:94) and Protein Sequence (SEQ ID NO:95)
of S11.4

atg ttc acc gtg ttt ctg ttg gtt gtc ttg gca acc acc gtc gtt ccc
Met Phe Thr Val Phe Leu Leu Val Val Leu Ala Thr Thr Val Val Pro
ttc aat tca gat cgt gat cca gca tta ggt ggc agg aat gct gca gcc
Phe Asn Ser Asp Arg Asp Pro Ala Leu Gly Gly Arg Asn Ala Ala Ala
ata gcg tct gac aag atc gct tcg acc ctc agg aga gga gga tgc tgt
Ile Ala Ser Asp Lys Ile Ala Ser Thr Leu Arg Arg Gly Gly Cys Cys
tct tat cct ccc tgt aac gtg tcc tat cca gaa att tgt ggt gga cga
Ser Tyr Pro Pro Cys Asn Val Ser Tyr Pro Glu Ile Cys Gly Gly Arg
cgc tgatgctcca ggaccctctg aaccacgacg t
Arg

TABLE 36

DNA Sequence (SEQ ID NO:96) and Protein Sequence (SEQ ID NO:97)
of S11.4A

atg ttc acc gtg ttt ctg ttg gtt gtc ttg gca acc acc gtc gtt tcc
Met Phe Thr Val Phe Leu Leu Val Val Leu Ala Thr Thr Val Val Ser
ttc aat tca gat cgt gca tta ggt ggc agg aat gct gca gcc aaa gcg
Phe Asn Ser Asp Arg Ala Leu Gly Gly Arg Asn Ala Ala Ala Lys Ala
tct gac aag atc gct tcg atc ctc ggg aga aga aga tgc tgt tct tat
Ser Asp Lys Ile Ala Ser Ile Leu Gly Arg Arg Arg Cys Cys Ser Tyr
cct ccc tgt aac gtg tcc tat cca gaa att tgt ggt gga cga cgc
Pro Pro Cys Asn Val Ser Tyr Pro Glu Ile Cys Gly Gly Arg Arg
tgatgctcca ggaccctctg aaccacgacg t

TABLE 37

DNA Sequence (SEQ ID NO:98) and Protein Sequence (SEQ ID NO:99)
of S11.8

atg ttc acc gtg ttt ctg ttg gtt gtc ttg gca acc acc gtc gtt tcc
Met Phe Thr Val Phe Leu Leu Val Val Leu Ala Thr Thr Val Val Ser
ttc aat tca gat cgt gca tta ggt ggc agg aat gct gca gcc aaa gcg
Phe Asn Ser Asp Arg Ala Leu Gly Gly Arg Asn Ala Ala Ala Lys Ala
tct gac aag atc gct tcg atc ctc ggg aga aga gca tgc tgt tct tat
Ser Asp Lys Ile Ala Ser Ile Leu Gly Arg Arg Ala Cys Cys Ser Tyr
cct ccc tgt aac gtg aac tat cca gaa att tgt ggt gga cga ggc
Pro Pro Cys Asn Val Asn Tyr Pro Glu Ile Cys Gly Gly Arg Gly
tgatgctcca ggaccctctg aaccacgacg t

TABLE 38

DNA Sequence (SEQ ID NO:100) and Protein Sequence
(SEQ ID NO:101) of P1.1

atg ttc acc gtg ttt ctg ttg gtt gtc ttg gca acc act gtc ggt tcc
Met Phe Thr Val Phe Leu Leu Val Val Leu Ala Thr Thr Val Gly Ser
ttc act tta gat cgt gca tct gat ggt agg gat gcc gca gcc aac gac
Phe Thr Leu Asp Arg Ala Ser Asp Gly Arg Asp Ala Ala Ala Asn Asp
aaa gcg act gac ctg atc gct ctg acc gcc agg aga gat cca tgc tgt
Lys Ala Thr Asp Leu Ile Ala Leu Thr Ala Arg Arg Asp Pro Cys Cys
tcc aat cct gtc tgt acc gtg cat aat cca cag att tgt ggt
Ser Asn Pro Val Cys Thr Val His Asn Pro Gln Ile Cys Gly
tgaagacgct gatgcttcag gaccctctga accacgacgt

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

Barnay, G. et al. (2000). J. Med. Chem.
Bitan, G. et al. (1997). J. Peptide Res. 49:421-426.
Blount, K. et al. (1992). Toxicon 30:835-842.
Bodanskyet al. (1966). Chem. Ind. 38:1597-98.
Craik, D. J. et al. (1991). Toxicon 39:43-60.
Cruz, L. J. et al. (1987). J. Biol. Chem. 260:9280-9288.
Goodman and Gilman [symbol] s The Pharmacological Basis of Therapeutics, 7th Ed., Section II (1985).
Gray, W. R. et al. (1981). J. Biol. Chem. 256:4734-4740.
Haack, J. A. et al. (1990). J. Biol. Chem. 265:6025-6029.
Horiki, K. et al. (1978). Chemistry Letters 165-68.
Hubry, V. et al. (1994). Reactive Polymers 22:231-241.
Kapoor (1970). J. Pharm. Sci. 59:1-27.
Kornreich, W. D. et al. (1986). U.S. Pat. No. 4,569,967.
Marshall, I. G. and Harvey, A. L. (1990). Toxicon 28:231-234.
McIntosh, J. M. et al. (1982). Arch. Biochem. Biophys. 218:329-334.
Mena, E. E. et al. (1990). Neurosci. Lett. 118:241-244.
Methoden der Organischen Chemie (Houben-Weyl): Synthese von Peptiden, E. Wunsch (Ed.), Georg Thieme Verlag, Stuttgart, Ger. (1974).
Myers, R. A. et al. (1991). Biochemistry 30:9370-9377.
Nishiuchi, Y. et al. (1993). Int. J. Pept. Protein Res. 42:533-538.
Nowak, L. et al. (1984). Nature 307:462-465.
Olivera, B. M. et al. (1984). U.S. Pat. No. 4,447,356.
Olivera, B. M. et al. (1985). Science 230:1338-1343.
Olivera, B. M. et al. (1996). U.S. Pat. No. 5,514,774.
Omstein, et al. (1993). Biorganic Medicinal Chemistry Letters 3:43-48.
Physicians Desk Reference, 48th Ed., pp. 689,758,1362, 1648 (1994).
Rivier, J. R. et al. (1978). Biopolymers 17:1927-38.
Rivier, J. R. et al. (1987). Biochem. 26:8508-8512.
Sambrook, J. et al. (1989). Molecular Cloning: A Laboratory Manual, 2nd Ed., Cold Spring Harbor Laboratory, Cold Spring Harbor, N.Y.
Schroder & Lubke (1965). The Peptides 1:72-75, Academic Press, NY.
Stewart and Young, Solid-Phase Peptide Synthesis, Freeman & Co., San Francisco, Calif. (1969).
Vale et al. (1978). U.S. Pat. No. 4,105,603.
Van de Steen, P. et al. (1998). Critical Rev. in Biochem. and Mol. Biol. 33:151-208.
Zafaralla, G. C. et al. (1988). Biochemistry 27:7102-7105.
Zhou L. M., et al. (1996). J. Neurochem. 66:620-628.
U.S. Pat. No. 3,972,859.
U.S. Pat. No. 3,842,067.
U.S. Pat. No. 3,862,925.
U.S. Pat. No. 4,190,674.
U.S. Pat. No. 4,179,507.
U.S. Pat. No. 4,508,715
U.S. Pat. No. 4,701,460.
U.S. Pat. No. 4,761,418.
U.S. Pat. No. 4,923,898.
U.S. Pat. No. 5,015,741.
U.S. Pat. No. 5,260,337.

What is claimed is:

1. A substantially pure α-conotoxin protein precursor comprising an amino acid sequence selected from the group of amino acid sequences set forth in Tables 2-38.

2. An isolated nucleic acid comprising a nucleic acid coding for an α-conotoxin precursor comprising an amino acid sequence selected from the group of amino acid sequences set forth in Tables 2-38.

3. The nucleic acid of claim 2 wherein the nucleic acid comprises a nucleotide sequence selected from the group of nucleotide sequences set forth in Tables 2-38 or their complements.

4. A method for providing musculoskeletal relaxation in a patient undergoing a surgical procedure requiring anesthesia which comprises administering an effective amount of an α-conotoxin peptide or a pharmaceutically acceptable salt thereof, said α-conotoxin peptide having the general formula I or general formula II:
Formula I: Xaa₁-Xaa₂-Xaa₃-Xaa₄-Cys-Cys-Xaa₅-Xaa₆-Xaa₇-Cys-Xaa₈-Xaa₉-Xaa₁₀ Xaa₁₁-Xaa₁₂-Xaa₁₃-Cys-Xaa₁₄-Xaa₁₅-Xaa₁₆-Xaa₁₇-Xaa₁₈-Xaa₁₉-Xaa₂₀-Xaa₂₁-Xaa₂₂-Xaa₂₃-Xaa₂₄-Xaa₂₅ (SEQ ID NO:1), wherein Xaa₁ is des-Xaa₁ or Gly; Xaa₂ is des-Xaa₂, Asn, Arg, Asp, Ser, Thr, Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₃ is des-Xaa₃, Gly, Glu or γ-carboxy-Glu (Gla); Xaa₄, is des-Xaa₄, Glu, Gla, Gln, pyro-Glu, Arg, Ile Tyr, mono-halo-Tyr, di-halo-Tyr, O-sulpho-Tyr, O-phospho-Tyr, nitro-Tyr, Cys, His, halo-His, any unnatural hydroxy containing amino acid (such as 4-hydroxymethyl-Phe, 4-hydroxyphenyl-Gly, 2,6-dimethyl-Tyr and 5-amino-Tyr), Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₅ is His, Asn or halo-His; Xaa₆ is Pro or hyroxy-Pro; Xaa₇ is Ala, Gly, Ser or Thr; Xaa₈ is Gly or Ala; Xaa₉ is Arg, Lys, Pro, hydroxy-Pro, Gly, Gln, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₁₀ is His, halo-His, Asn, Lys, Tyr, mono-halo-Tyr, di-halo-Tyr, O-sulpho-Tyr, O-phospho-Tyr, nitro-Tyr, N-methy-Lys, N,N-dimethyl-Lys, N,N,N-trimethyl-Lys, Arg, homoarginine, ornithine or any unnatural basic amino acid (such as N-1-(2-pyrazolinyl)-Arg); Xaa₁₁ is Tyr, Phe, mono-halo-Tyr, di-halo-Tyr, O-sulpho-Tyr, O-phospho-Tyr, nitro-Tyr, any unnatural hydroxy containing amino acid (such as 4-hydroxymethyl-Phe, 4-hydroxyphenyl-Gly, 2,6-dimethyl-Tyr and 5-amino-Tyr), Trp (D or L), halo-Trp, neo-Trp, or any unnatural aromatic amino acid (such as nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc); Xaa₁₂ is Ile, Ser, Thr, Asp, Gly, Asn, Glu, Gla or Val; Xaa₁₃ is des-Xaa₁₃, Lys, Arg, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₁₄ is des-Xaa₁₄, Gly, Lys, Arg, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₁₅ is des-Xaa₁₅, Gly, Thr, Ser, His, halo-His, Lys, Arg, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₁₆ is des-Xaa₁₆, Ser or Thr; Xaa₁₇ is des-Xaa₁₇ or Cys; Xaa₁₈ is des-Xaa₁₈, Ser or Thr; Xaa₁₉ is des-Xaa₁₉, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₂₀ is des-Xaa₂₀, Thr, Ser, Pro or hydroxy-Pro; Xaa₂₁ is des-Xaa₂₁, Leu, Ser or Thr; Xaa₂₂ is des-Xaa₂₂, Glu or Gla; Xaa₂₃ is des-Xaa₂₃, Pro or hydroxy-Pro; Xaa₂₄ is des-Xaa₂₄, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₂₅ is des-Xaa₂₅, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg); and the C-terminus may contain a free carboxyl group or an amide group;
Formula II: Xaa₁-Xaa₂-Xaa₃-Cys-Cys-Xaa₄-Xaa₅-Xaa₆-Xaa₇-Cys-Xaa₈-Xaa₉-Xaa₁₀-Xaa₁₁-Xaa₆-Xaa₁₂-Ile-Cys-Xaa₁₃-Xaa₁₄-Xaa₁₅ (SEQ ID NO:2), wherein, Xaa₁ is des-Xaa₁, Arg, Ser, Thr, Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₂ is des-Xaa₂, Asp, Gly, Leu, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₃ is des-Xaa₃, Pro, hydroxy-Pro, Ala, Gly or Leu; Xaa₄ is Tyr, Ser, Thr, mono-halo-Tyr, di-halo-Tyr, O-sulpho-Tyr, O-phospho-Tyr, nitro-Tyr or any unnatural hydroxy containing amino acid (such as 4-hydroxymethyl-Phe, 4-hydroxyphenyl-Gly, 2,6-dimethyl-Tyr and 5-amino-Tyr); Xaa₅ is His, Asn, Ile, Tyr, halo-His, mono-halo-Tyr, di-halo-Tyr, O-sulpho-Tyr, O-phospho-Tyr or nitro-Tyr; Xaa₆ is Pro or hydroxy-Pro; Xaa₇ is Thr, Ala, Val, Ser, Pro or hydroxy-Pro; Xaa₈ is Asn, Thr, Ser, Lys, Arg, ornithine, homoargine, N-methy-Lys, N,N-dimethyl-Lys, N,N,N-trimethyl-Lys or any unnatural basic amino acid (such as N-1-(2-pyrazolinyl)-Arg); Xaa₉ is Met, Val, Ala, Leu or Ile; Xaa₁₀ is Ser, Thr, Asn, His or halo-His; Xaa₁ is Asn, Tyr, mono-halo-Tyr, di-halo-Tyr, O-sulpho-Tyr, O-phospho-Tyr, nitro-Tyr, or any unnatural hydroxy containing amino acid (such as 4-hydroxymethyl-Phe, 4-hydroxyphenyl-Gly, 2,6-dimethyl-Tyr and 5-amino-Tyr); Xaa₁₂ is Glu, γ-carboxy-Glu (Gla), Gln or Asp; Xaa₁₃ is des-Xaa₁₃ or Gly; Xaa₁₄ is des-Xaa₁₄ or Gly; Xaa₁₅ is des-Xaa₁₅, Arg, Lys, ornithine, homoargine, N-methy-Lys, N,N-dimethyl-Lys, N,N,N-trimethyl-Lys or any unnatural basic amino acid (such as N-1-(2-pyrazolinyl)-Arg) and the C-terminus may contain a free carboxyl group or an amide group.

5. The method of claim 4, wherein said α-conotoxin peptide is selected from the group consisting of:
Xaa₁-Cys-Cys-Asn-Xaa₂-Ala-Cys-Gly-Arg-His-Xaa₃-Ser-Cys-Xaa₄-Gly (SEQ ID NO:3);
Asn-Gly-Arg-Cys-Cys-His-Xaa₂-Ala-Cys-Gly-Xaa₄-His-Phe-Ser-Cys (SEQ ID NO:4);
Gly-Arg-Gly-Arg-Cys-Cys-His-Xaa₂-Ala-Cys-Gly-Xaa₂-Asn-Xaa₃-Ser-Cys (SEQ ID NO:5);
Cys-Cys-His-Xaa₂-Ala-Cys-Gly-Arg-Xaa₄-Xaa₃-Asn-Cys (SEQ ID NO:6);
Cys-Cys-Cys-Asn-Xaa₂-Ala-Cys-Gly-Xaa₂-Asn-Xaa₃-Gly-Cys-Gly-Thr-Ser-Cys-Ser-Arg-Xaa₂-Ser-Xaa₁-Xaa₂-Arg-Arg (SEQ ID NO:7);
Asn-Gly-His-Cys-Cys-His-Xaa₂-Ala-Cys-Gly-Gly-Xaa₄-Xaa₃-Val-Xaa₄-Cys (SEQ ID NO:8);
Asn-Gly-Arg-Cys-Cys-His-Xaa₂-Ala-Cys-Gly-Gly-Xaa₄-Xaa₃-Val-Xaa₄-Cys (SEQ ID NO:9);
Asn-Gly-Arg-Cys-Cys-His-Xaa₂-Ala-Cys-Gly-Xaa₄-His-Phe-Ile-Cys (SEQ ID NO:10);
Asn-Gly-Arg-Cys-Cys-His-Xaa₂-Ala-Cys-Gly-Xaa₄-His-Phe-Ser-Cys (SEQ ID NO:11);
Asn-Gly-Arg-Cys-Cys-His-Xaa₂-Ser-Cys-Gly-Arg-Xaa₄-Xaa₃-Asn-Cys (SEQ ID NO:12);
Asn-Gly-Arg-Cys-Cys-His-Xaa₂-Ala-Cys-Ala-Arg-Xaa₄-Xaa₃-Asn-Cys (SEQ ID NO:13);
Asn-Xaa₁-Arg-Cys-Cys-His-Xaa₂-Ala-Cys-Ala-Arg-Xaa₄-Xaa₃-Asn-Cys (SEQ ID NO:14);
Asp-Gly-Arg-Cys-Cys-His-Xaa₂-Ala-Cys-Gly-Gln-Asn-Xaa₃-Ser-Cys (SEQ ID NO:15);
Asp-Gly-Arg-Cys-Cys-His-Xaa₂-Ala-Cys-Ala-Xaa₄-His-Phe-Asn-Cys (SEQ ID NO:16);
Asn-Gly-Arg-Cys-Cys-His-Xaa₂-Ala-Cys-Ala-Xaa₄-Asn-Xaa₃-Ser-Cys (SEQ ID NO:17);
Asn-Gly-Arg-Cys-Cys-His-Xaa₂-Ala-Cys-Ala-Arg-Xaa₄-Xaa₃-Ser-Cys (SEQ ID NO:18);
Xaa₅-Cys-Cys-Asn-Xaa₂-Ala-Cys-Gly-Xaa₂-Xaa₄-Xaa₃-Ser-Cys (SEQ ID NO:19);
Xaa₅-Cys-Cys-His-Xaa₂-Ala-Cys-Gly-Xaa₄-Xaa₄-Xaa₃-Asn-Cys (SEQ ID NO:20);
Ser-Gly-Arg-Cys-Cys-His-Xaa₂-Ala-Cys-Gly-Arg-Xaa₄-Xaa₃-Asn-Cys (SEQ ID NO:21);
Arg-Asp-Xaa₂-Cys-Cys-Ser-Asn-Xaa₂-Val-Cys-Thr-Val-His-Asn-Xaa₂-Gln-Ile-Cys (SEQ ID NO:22);
Arg-Ala-Cys-Cys-Ser-Xaa₃-Xaa₂-Xaa₂-Cys-Asn-Val-Asn-Xaa₃-Xaa₂-Xaa₁-Ile-Cys (SEQ ID NO:23);
Gly-Gly-Cys-Cys-Ser-Xaa₃-Xaa₂-Xaa₂-Cys-Asn-Val-Ser-Xaa₃-Xaa₂-Xaa₁-Ile-Cys (SEQ ID NO:24);
Cys-Cys-Ser-Xaa₃-Xaa₂-Xaa₂-Cys-Asn-Val-Ser-Xaa₃-Xaa₂-Xaa₁-Ile-Cys (SEQ ID NO:25);
Ala-Cys-Cys-Ser-Xaa₃-Xaa₂-Xaa₂-Cys-Asn-Val-Asn-Xaa₃-Xaa₂-Xaa₁-Ile-Cys-Gly-Gly-Arg (SEQ ID NO:26); and
Ser-Leu-Ieu-Cys-Cys-Thr-Ile-Xaa₂-Ser-Cys-Xaa₄-Ala-Ser-Xaa₃-Xaa₂-Asp-Ile-Cys (SEQ ID NO:27),
wherein Xaa₁ is Glu or γ-carboxy-glutamate (Gla); Xaa₂ is Pro or hydroxy-Pro; Xaa₃ is Tyr, mono-halo-Tyr, di-halo-Tyr, O-sulpho-Tyr, O-phospho-Tyr or nitro-Tyr; Xaa₄ is Lys, N-methyl-Lys, N,N-dimethyl-Lys or N,N,N-trimethyl-Lys; Xaa₅ is Gln or pyro-Glu; and the C-terminus contains a carboxyl or amide group.
